SEQUENCE LISTING (6 sequences)

SEQ ID NO: 1 - DNA, 283 bp, Homo sapiens
ggccgggcgcggtggctcacgcctgtaatcccagcactttgggaggccgaggcgggcggatcacctgaggtcaggagttcgagaccagcctggccaacatggtgaaaccccgtctctactaaaaatacaaaaattagccggggcgtggtggcgcgcgcctgtaatcccagctactcgggaggctgaggcaggagaatcgcttgaacccgggaggcggaggttgcagtgagccgagatcgcgccactgcactccagcctgggcgacagagcgagactccgtctc

SEQ ID NO: 2 - DNA, 281 bp, Homo sapiens
ggccgggcgcggtggctcacgcctgtaatcccagcactttgggaggccgaggcgggcggatcacgaggtcaggagttcgagaccagcctggccaacatggtgaaaccccgtctctactaaaaatacaaaaattagccggggcgtggtggcgcgcgcctgtaatcccagctactcgggaggctgaggcaggagaatcgcttgaacccgggaggcggaggttgcagtgagccgagatcgcgccactgcactccagcctgggcgacagagcgagactccgtctc

SEQ ID NO: 3 - DNA, 282 bp, Homo sapiens
ggccgggcgcggtggctcacgcctgtaatcccagcactttgggaggccgaggcgggcggatcacgaggtcaggagatcgagaccatcctggctaacacggtgaaaccccgtctctactaaaaatacaaaaaattagccggggcgtggtggcgggcgcctgtagtcccagctactcgggaggctgaggcaggagaatggcgtgaacccgggaggcgcaggttgcagtgagccgagatcgcgccactgcactccagcctgggcgacagagcgagactccgtctc

SEQ ID NO: 4 - DNA, 282 bp, Homo sapiens
ggccgggcgcggtggctcacgcctgtaatcccagcactttgggaggccgaggcgggcggatcacgaggtcaggagatcgagaccatcccggctaaaacggtgaaaccccgtctctactaaaaatacaaaaaattagccggggcgtagtggcgggcgcctgtagtcccagctacttgggaggctgaggcaggagaatggcgtgaacccgggaggcgcaggttgcagtgagccgagatcccgccactgcactccagcctgggcgacagagcgagactccgtctc

SEQ ID NO: 5 - DNA, 281 bp, Homo sapiens
ggccgggcgcggtggctcacgcctgtaatcccagcactttgggaggccgaggcgggcggatcacgaggtcaggagatcgagaccatcccggctaaaacggtgaaaccccgtctctactaaaactacaaaaaatagccggggcgtagtggcgggcgcctgtagtcctagctacttgggaggctgaggcaggagaatggcgtgaacccgggaggcgcaggttgcagtgagccgagatcccgccactgcactccagcctgggcgacagagcgagactccgtctc

SEQ ID NO: 6 - DNA, 290 bp, Homo sapiens
ggccgggcgcggtggctcacgcctgtaatcccagcactttgggaggccgaggcgggtggatcatgaggtcaggagatcgagaccatcctggctaacaaggtgaaaccccgtctctactaaaaatacaaaaaattagccggggcgcggtggcgggcgcctgtagtcccagctactcgggaggctgaggcaggagaatggcgtgaacccgggaggcgcaggttgcagtgagccgagattgcgccactgcagtccagcagtccggcctgggcgacagagcgagactccgtctc

(19) United States
(12) Patent Application Publication
(10) Pub. No.: US 2005/0186589 A1
(43) Pub. Date: Aug. 25, 2005

(54) Interspersed repetitive element RNAs as substrates, inhibitors and delivery vehicles for RNAi

(75) Inventors: Timothy F. Kowalik, Princeton, MA (US); Bradford M. Stadler, Marlborough, MA (US)

Correspondence Address: LAHIVE & COCKFIELD, LLP., 28 STATE STREET, BOSTON, MA, 02109, US

(73) Assignee: UNIVERSITY OF MASSACHUSETTS, Boston, MA (US)

(21) Appl. No.: 10/984,180

(22) Filed: Nov. 8, 2004

Related U.S. Application Data
(60) Provisional application No. 60/518,423, filed on Nov. 7, 2003.

(51) Int. Cl. A61K 48/00; C12Q 1/68; A01N 43/04

(52) U.S. Cl. 435/6; 514/44

(57) ABSTRACT

The present invention provides methods for identifying druggable targets in assays that feature compositions, cells and/or organisms having interspersed repetitive element (IRE) RNAs and an RNA interference (RNAi) pathway. Methods for identifying therapeutic agents and creating vaccines are also featured. The invention further provides methods for inhibiting RNAi involving IRE RNAs or inhibitory derivatives thereof. The invention also provides compositions for delivering siRNA and miRNA molecules derived from IRE loci and methods of use thereof. Therapeutic methods are also featured.

### RELATED APPLICATIONS

This patent application claims the benefit of U.S. Provisional Patent Application Ser. No. 60/518,423, entitled “Interspersed Repetitive Element RNAs as Substrates, Inhibitors and Delivery Vehicles for RNAi”, filed Nov. 7, 2003. The entire contents of the above-referenced provisional patent application are incorporated herein by this reference.

### BACKGROUND OF THE INVENTION

RNAs that do not function as messenger RNAs, transfer RNAs or ribosomal RNAs, are collectively termed non-coding RNAs (ncRNAs). ncRNAs can range in size from 21-25 nucleotides (nt) up to >10,000 nt, and estimates for the number of ncRNAs per genome range from hundreds to thousands. The functions of ncRNAs, although just beginning to be revealed, appear to vary widely from the purely structural to the purely regulatory, and include effects on transcription, translation, mRNA stability and chromatin structure (G. Storz, Science (2002) 296:1260-1262). Two recent pivotal discoveries have placed ncRNAs in the spotlight: the identification of large numbers of very small ncRNAs of 20-24 nucleotides in length, termed micro RNAs (miRNAs), and the relationship of these miRNAs to intermediates in a eukaryotic RNA silencing mechanism known as RNA interference (RNAi).

RNA silencing refers to a group of sequence-specific, RNA-targeted gene-silencing mechanisms common to animals, plants, and some fungi, wherein RNA is used to target and destroy homologous mRNA, viral RNA, or other RNAs. RNA silencing was first observed in plants, where it was termed posttranscriptional gene silencing (PTGS). Researchers, trying to create more vividly purple flowers, introduced an extra copy of the gene conferring purple pigment. Surprisingly, the researchers discovered that the purple-conferring genes were switched off, or cosuppressed, producing white flowers. A similar phenomenon observed in Fungi was termed quelling. These phenomena were subsequently found to be related to a process in animals called RNA interference (RNAi). In RNAi, experimentally introduced double-stranded RNA (dsRNA) leads to loss of expression of the corresponding cellular gene. A key step in the molecular mechanism of RNAi is the processing of dsRNA by the ribonuclease Dicer into short dsRNAs, called small interfering RNAs (siRNAs), of ˜21-23 nt in length and having specific features including 2 nt 3′-overhangs, a 5′-phosphate group and 3′-hydroxyl group. siRNAs are incorporated into a large nucleoprotein complex called RNA-induced silencing complex (RISC). A distinct ribonuclease component of RISC uses the sequence encoded by the antisense strand of the siRNA as a guide to find and then cleave mRNAs of complementary sequence. The cleaved mRNA is ultimately degraded by cellular exonucleases. Thus, in PTGS, quelling, and RNAi, the silenced gene is transcribed normally into mRNA, but the mRNA is destroyed as quickly as it is made. In plants, it appears that PTGS evolved as a defense strategy against viral pathogens and transposons. While the introduction of long dsRNAs into plants and invertebrates initiates specific gene silencing (3,4), in mammalian cells, long dsRNA induces the potent translational inhibitory effects of the interferon response (8). Short dsRNAs of <30 bp, however, evade the interferon response and are successfully incorporated into RISC to induce RNAi (9).

Another group of small ncRNAs, called micro RNAs (miRNAs), are related to the intermediates in RNAi and appear to be conserved from flies to humans (2, 12, 13). miRNAs are transcribed first as a long primary transcript (pri-miRNAs), in some cases as miRNAs clusters, and recent evidence indicates that these transcripts are initially processed by the ribonuclease Drosha to ˜70 nt RNA precursors (pre-miRNAs) having a predicted stem-loop structure (31). The ribonuclease Dicer then cleaves these pre-miRNAs to produce ˜20-24 nt miRNAs that function as single-stranded RNAi mediators (4, 10). These small transcripts have been proposed to play a role in development, apparently by suppressing target genes to which they have some degree of complementarity. The founding members of miRNAs, lin-4 and let-7, exert their control of gene expression by binding to non-identical sequences in the 3′ UTR of mRNA, thereby preventing mRNA translation (17). In recent studies, however, miRNAs bearing perfect complementarity to a target RNA could function as siRNAs to specifically degrade the target sequences (14, 15). Thus, the degree of complementarity between an miRNA and its target may determine whether the miRNA acts as a translational repressor or as a guide to induce mRNA cleavage.

The discovery of miRNAs as endogenous small regulatory ncRNAs may represent the tip of the iceberg, with other groups of regulatory ncRNAs still to be discovered. In addition to post-transcription silencing activity, the components of the RNAi pathway have been implicated to function in mechanisms of transcriptional gene silencing (TGS) and heterochromatic silencing. Most notably, evidence from plants and Schizosaccharomyces pombe illustrate the involvement of the RNAi pathway in promoter methylation and the formation and maintenance of heterochromatin (32, 33). It is possible that additional groups of ncRNAs may also function through the RNAi pathway. Such ncRNAs would provide useful reagents and strategies for modulating gene expression and developing novel therapeutics.

### SUMMARY OF THE INVENTION

The present invention is based in part on the observation that the secondary structure of interspersed repetitive element (IRE) RNAs, and in particular Alu SINE RNAs, is similar to that of endogenous cellular pri-mRNAs or pre-miRNAs. Pri-mRNAs are initially processed by the ribonuclease Drosha to stem-loop precursors (pre-miRNAs) which have a form accessible to the ribonuclease Dicer. Pre-miRNAs are then processed by Dicer via the RNAi pathway to generate ˜21-23 nt RNA product. IRE RNAs, e.g., Alu RNAs are proposed to be similarly processed by Drosha and/or Dicer into miRNAs or siRNAs, which in turn may be incorporated into a Dicer (or an orthologue or homologue thereof) or RISC complex to function as substrates and/or inhibitors of the RNAi pathway.

Accordingly, the present invention features interspersed repetitive element (IRE) RNAs, e.g., Alu RNAs (or derivatives thereof) for use as mediators of RNAi. In one embodiment, the IRE RNAs (or derivatives thereof) are activators of RNAi. Also featured are IRE RNAs (or derivatives thereof) for use as inhibitors of RNAi. Also featured are methods for identifying druggable targets mediated by the IRE RNAs (or derivatives thereof). Such targets are further useful in drug discovery methodologies. Also featured are expression cassettes and vectors (e.g., plasmid based or virus-derived vectors), the cassettes and/or vectors including IRE RNA loci modified to deliver miRNA- and siRNA-like molecules. Further featured are methods of enhancing exogenous gene expression mediated by IRE RNAs (or derivatives thereof).

Other features and advantages of the invention will be apparent from the following detailed description, and from the claims.

### BRIEF DESCRIPTION OF THE DRAWINGS

FIG. 1 is the predicted secondary structure of Alu RNA.

FIG. 2 depicts the results of Northern analysis of Alu RNA cleavage products in heat shocked or adenovirus infected cells.

FIG. 3A-D depicts a typical human Alu element structure and its retroposition. FIG. 3A shown a typical Alu element, and an Alu RNA is shown in FIG. 3B. Insertion and reverse transcription of Alu RNA is depicted in FIG. 3C and second-site nick and ligation is shown in FIG. 3D.

FIG. 4 depicts an alignment of Alu-subfamily consensus sequences.

### DETAILED DESCRIPTION OF THE INVENTION

The present invention is based, at least in part, on the observation that RNA transcripts produced from interspersed repetitive elements (IREs), e.g., short interspersed elements (SINEs), and in particular Alu RNAs, bear a striking resemblance to pri-miRNAs or pre-miRNAs. Pri-miRNAs are long primary transcripts encoding miRNAs that are initially processed in the nucleus by the nuclear RNase III enzyme Drosha (31) into pre-miRNAs. Pre-miRNAs are complex, double-stranded precursor RNA molecules characterized by key structural features such as stem loops and bulges (4, 10). Pre-miRNAs are processed by the cytoplasmic ribonuclease Dicer to generate ˜21-23 nt RNA products termed miRNAs.

IREs represent a large group of mobile or transposable elements which are highly abundant in the genome. SINEs, e.g., Alu SINEs, represent a particularly abundant group of IREs. Other IREs include long interspersed elements (LINEs) and long terminal repeat (LTR) retrotransposons. To date, the function of IREs, and in particular, Alu RNAs, is largely unknown. Given the similar structure between, at least, Alu RNAs and pri- and/or pre-miRNAs, IRE RNAs (e.g., Alu RNAs) (or derivatives thereof) are proposed to be processed by the RNAi machinery in a manner similar to the processing of pri-miRNA into pre-miRNA by Drosha, and of pre-miRNA into miRNAs by Dicer. While not wishing to be bound by theory, considering that IREs reside in the host genome, it is possible that the structured RNAs produced from the IREs are initially processed by Drosha in the nucleus prior to further processing by the cytoplasmic enzyme Dicer.

Based on the observations set forth herein, IRE RNAs (e.g., Alu RNAs) are proposed to act as precursors for cleavage by Drosha and/or Dicer to produce miRNA-like or siRNA-like molecules that regulate gene expression during times of cellular insult. Cellular and/or viral genes whose RNA expression is modulated by IRE RNAs (e.g., Alu RNAs) make attractive druggable targets, e.g., for therapeutic anti-viral strategies as well as novel ways to modulate host homeostasis.

IRE RNAs (e.g., Alu RNAs) are further proposed to act as inhibitors of RNAi by competing with other substrates for interaction with components of the RNAi pathway, e.g. Dicer, or components of RISC, thus preventing processing of other potential RNAi triggers, including host miRNA precursors and exogenous RNA species, e.g., viral RNA species. Such inhibition could represent a natural cellular defense mechanism. Enhancing the RNAi inhibition by IRE RNAs (e.g., Alu RNAs) provides novel approaches for the design of therapeutic agents. IRE RNAs are therefore useful in methods of inhibiting RNAi.

It is further proposed that IRE loci (e.g., Alu loci) can be modified to express miRNA- and siRNA-like molecules directed to selected target RNAs, thereby providing a novel siRNA/miRNA transduction system.

It is also within the scope of the present invention to use IRE RNAs in methods of enhancing exogenous gene expression.

Based at least in part on the above observations, the invention features, in a first aspect, methods for identifying genes whose expression is modulated by IRE RNAs (e.g., Alu RNAs). In an exemplary aspect, the genes identified are involved in important cellular processes, for example, in the response to cell stress. Accordingly, the genes make desirable targets for drug discovery (i.e., druggable targets).

Accordingly, in one embodiment, the invention provides a method for identifying a druggable target, involving the steps of: (a) obtaining an assay composition comprising an RNAi pathway molecule and an interspersed repetitive element (IRE) RNA; and (b) assaying for expression of a candidate RNA; wherein a change in expression of the candidate RNA indicates that a gene or protein corresponding to the RNA is a druggable target. In a preferred embodiment of this aspect, the assay composition is a cell extract, e.g., a mammalian cell extract.

In a related embodiment, the invention provides a method for identifying a druggable target, involving the steps of: (a) obtaining a cell or organism comprising an RNAi pathway and an interspersed repetitive element (IRE) RNA; (b) assaying for expression of a candidate RNA; wherein a change in expression of the candidate RNA indicates that a gene or protein corresponding to the RNA is a druggable target.

In preferred embodiments, the druggable target in an antiviral drug target. In other embodiments, the change in expression of the candidate RNA is a decrease in the expression of the candidate RNA.

In one embodiment, these methods further involve the step of preselecting the candidate RNA. In an exemplary embodiment, the preselection step involves determining a sufficient degree of sequence identity between the interspersed repetitive element (IRE) RNA and the candidate RNA, e.g., the IRE RNA and the candidate RNA share at least 60%, 70%, 80%, or 90% sequence identity. In another embodiment, the preselection step involves selecting the candidate RNA based on its encoding a gene or protein having a desired cellular function, e.g., maintenance of cellular homeostasis, maintenance of differentiation, regulation of cell cycle, regulation of glucose metabolism, promotion of apoptosis and inhibition of apoptosis. In another embodiment, the preselection step includes selecting the candidate RNA based on its comprising an interspersed repetitive element (IRE) sequence or portion thereof.

In one embodiment, the candidate RNA is a mRNA, e.g., a mRNA which encodes a cellular protein or a viral protein. In another embodiment, the candidate RNA is a ncRNA regulating gene expression. In one embodiment, the candidate RNA is transcribed from a gene comprising an interspersed repetitive element (IRE) or portion thereof.

The invention features, in a related aspect, a druggable target identified according to the methods set forth above.

The invention features, in a second aspect, methods for identifying therapeutic agents, wherein the agents modulate the expression or activity of a druggable target identified through the methods of the invention, or which inhibit the generation of the siRNA or miRNA.

Accordingly, in one embodiment, the invention provides a method for identifying a therapeutic agent, involving assaying a test agent for activity against a druggable target of the invention. In another embodiment, a method for identifying a therapeutic agent involves assaying a test agent for the ability to stimulate expression or activity of a druggable target of the invention. In yet another embodiment, a method for identifying a therapeutic agent involves assaying a test agent for the ability to inhibit an interaction between a druggable target of the invention and a corresponding interspersed repetitive element RNA.

In one embodiment, the invention provides a method for identifying a therapeutic agent, involving: (a) contacting a cell with a test agent, said cell comprising an RNAi pathway and an interspersed repetitive element RNA, wherein said RNAi pathway generates a siRNA or miRNA from said interspersed repetitive element RNA; (b) detecting an indicator of said siRNA or miRNA; wherein an agent is identified based on its ability to inhibit the generation of said siRNA or miRNA.

In a related embodiment, a method is provided for identifying a therapeutic agent, involving: (a) contacting an assay composition with a test agent, wherein said assay composition comprises an RNAi pathway molecule and an IRE RNA, wherein said RNAi pathway molecule generates a siRNA or miRNA from said IRE RNA; and (b) detecting an indicator of said siRNA or miRNA; wherein an agent is identified based on its ability to inhibit the generation of said siRNA or miRNA.

In another embodiment, the invention provides a method of treating a disease or disorder in a subject, involving administering to the subject a therapeutically effective dose of an agent or composition of the invention, such that the disease or disorder is treated. Preferably, the organism or subject is a eukaryotic organism, e.g., a mammal, and preferably a human.

The invention further features, in a third aspect, methods for inhibiting RNAi involving an IRE RNA. In a related aspect, the invention provides methods for identifying a therapeutic agent, wherein the agent promotes the inhibition by IRE RNA of either an RNAi pathway or the activity of RNAi molecules.

Accordingly, the invention features, in one embodiment, a method for inhibiting RNAi in a cell, involving introducing into the cell an interspersed repetitive element (IRE) RNA or inhibitory derivative thereof, such that RNAi in the cell is inhibited.

In a related embodiment, a method is provided for inhibiting the incorporation of a siRNA or miRNA into a cellular Dicer or RISC complex, involving introducing into the cell an isolated interspersed repetitive element (IRE) RNA or inhibitory derivative thereof, such that incorporation of the siRNA or miRNA into the complex is inhibited.

In one embodiment, the invention provides a method for identifying a therapeutic agent, involving: (a) contacting a cell with a test agent, said cell comprising an RNAi pathway and an interspersed repetitive element (IRE) RNA, wherein the ribonucleotide inhibits the RNAi pathway; and (b) detecting an indicator of the RNAi pathway; wherein an agent is identified based on its ability to promote inhibition of the RNAi pathway.

In a related embodiment, the invention provides a method for identifying a therapeutic agent, involving: (a) contacting an assay composition with a test agent, wherein said assay composition comprises a RNAi pathway molecule and an interspersed repetitive element (IRE) RNA which inhibits the activity of said RNAi pathway molecule; and (b) detecting activity of said RNAi pathway molecule; wherein said agent is identified based on its ability to further inhibit activity of said RNAi pathway molecule.

In another related embodiment, the invention provides a method for identifying a therapeutic agent, comprising : (a) contacting an assay composition with a test agent, wherein said assay composition comprises an interspersed repetitive element (IRE) RNA and a RNAi pathway molecule capable of interacting with or altering the IRE RNA; (b) detecting the ability of the RNAi pathway molecule to interact with or alter the IRE RNA; wherein said agent is identified based on its ability to modulate the interaction of the IRE RNA with RNAi pathway molecule or alteration of the IRE RNA by the RNAi pathway molecule.

In preferred embodiments, the RNAi pathway molecule is a RISC component or Dicer (or a homologue thereof).

The invention features, in a fourth aspect, vectors and cassettes for delivering siRNA or miRNA molecules from an IRE locus. In an exemplary aspect, the vector is a plasmid or is derived from a virus.

Accordingly, the invention provides, in various embodiments, a vector or cassette for delivering a siRNA or miRNA, comprising an interspersed repetitive element (IRE) locus that has been modified to comprise a nucleotide sequence that encodes a siRNA or miRNA precursor. In certain embodiment, the vectors and cassettes further include either a polymerase III promoter or a promoter endogenous to the IRE locus operably linked to the nucleotide sequence.

In preferred embodiments, the sequence of the miRNA or siRNA molecule is sufficiently complementary to a RNA sequence to mediate degradation of said RNA sequence, to inhibit translation of said RNA sequence, or to a RNA sequence to induce chromatin silencing of a DNA sequence encoding the RNA sequence.

In an exemplary embodiment, the invention provides a vector that expresses a siRNA or miRNA from an interspersed repetitive element (IRE) locus. In a preferred embodiment, the siRNA or miRNA is exogenous. The invention further provides a composition comprising a vector of this aspect and a pharmaceutically acceptable carrier.

The invention further provides, in a related aspect, methods for inducing gene silencing, e.g., posttranscriptional gene silencing or transcriptional gene silencing, involving administering compositions comprising vectors of the invention.

Accordingly, in one embodiment, the invention provides a method for targeting degradation of a RNA in a subject, involving administering to the subject a composition of this aspect of the invention, wherein the siRNA or miRNA has a ribonucleotide sequence having sufficient complementarity to the target RNA, such that the targets are degraded. In a related embodiment, the invention provides a method for inhibiting translation of a RNA in a subject, involving administering to the subject a composition of the invention, wherein the siRNA or miRNA has a ribonucleotide sequence having sufficient complementarity to the target RNA, such that the targets are translationally inhibited. In preferred embodiments, the siRNA or miRNA has a ribonucleotide sequence sufficiently complementary to a mutant allelic target RNA, such that the mutant allelic target is degraded or is translationally inhibited.

In another embodiment, the invention provides a method for targeting a DNA sequence for chromatin silencing in a subject, comprising administering to the subject a composition of the invention, wherein the siRNA or miRNA has a ribonucleotide sequence having sufficient complementarity to a RNA encoded by the target DNA sequence such that the target DNA sequence is chromatically silenced. In a preferred embodiment, at least one siRNA or miRNA has a ribonucleotide sequence sufficiently complementary to a RNA encoded by a mutant allelic target DNA sequence, such that the mutant allelic target DNA sequence is chromatically silenced.

In preferred embodiments, the interspersed repetitive element (IRE) locus becomes integrated in the genome of the subject. Preferably, integration is at a genomic IRE locus, e.g., where the genomic IRE locus is present in an untranslated region of the genome.

In another embodiment, the invention provides a vaccine comprising the vector, wherein at least one siRNA or miRNA targets either a viral gene product or a cellular gene.

The invention provides, in yet another aspect, a method for upregulating exogenous gene expression in a cell, involving introducing into a cell having an RNAi pathway an interspersed repetitive element (IRE) RNA, wherein the IRE RNA is a substrate or inhibitor of the RNAi pathway, such that exogenous gene expression is upregulated.

In one embodiment, the invention provides a method for efficiently introducing an exogenous gene into a cell, comprising introducing into a cell having an RNAi pathway the exogenous gene and an interspersed repetitive element (IRE) RNA, wherein the IRE RNA is a substrate or inhibitor of the RNAi pathway, such that the exogenous gene is efficiently introduced.

In various embodiments of the invention, the cell is a eukaryotic cell, e.g., a plant cell or an insect cell. In a preferred embodiment, the cell is a mammalian cell, e.g., a murine cell, an avian cell, or a human cell. In one embodiment, the cell is present in an organism, preferably a human subject.

In various embodiments of the invention, the interspersed repetitive (IRE) element is a short interspersed element (SINE), a long interspersed element (LINE), or a long terminal repeat (LTR)-retrotransposon. In a preferred embodiment, the short interspersed element is an Alu element.

In various embodiments of the invention, the interspersed repetitive element RNA is expressed from a virus, a vector or a cassette.

In various embodiments of the invention, the invention provides an agent identified by any of the methods of the invention. The invention further provides a composition comprising the agents identified by any of the methods of the invention and a pharmaceutically acceptable carrier.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

I. Definitions

So that the invention may be more readily understood, certain terms are first defined.

The term “target gene”, as used herein, refers to a gene intended for downregulation via RNA interference (“RNAi”). The term “target protein” refers to a protein intended for downregulation via RNAi. The term “target RNA” refers to an RNA molecule intended for degradation by RNAi. The term “target RNA” includes both non-coding RNA molecules (transcribed from a DNA but not encoding polypeptide sequence) and coding RNA molecules (i.e., mRNA molecules). A “target RNA” is also referred to herein as a “transcript”.

The term “RNA interference” or “RNAi”, as used herein, refers generally to a sequence-specific or selective process by which a target molecule (e.g., a target gene, protein or RNA) is downregulated. In specific embodiments, the process of “RNA interference” or “RNAi” features degradation of RNA molecules, e.g., RNA molecules within a cell, said degradation being triggered by an RNA agent. Degradation is catalyzed by an enzymatic, RNA-induced silencing complex (RISC). RNAi occurs in cells naturally to remove foreign RNAs (e.g., viral RNAs). Natural RNAi proceeds via fragments cleaved from free dsRNA which direct the degradative mechanism to other similar RNA sequences. Alternatively, RNAi can be initiated by the hand of man, for example, to silence the expression of target genes.

The term “RNA agent”, as used herein, refers to an RNA (or analog thereof), having sufficient sequence complementarity to a target RNA (i.e., the RNA being degraded) to direct RNAi. A RNA agent having a “sequence sufficiently complementary to a target RNA sequence to direct RNAi” means that the RNA agent has a sequence sufficient to trigger the destruction of the target RNA by the RNAi machinery (e.g., the RISC complex) or process. A RNA agent having a “sequence sufficiently complementary to a target RNA sequence to direct RNAi” is also intended to mean that the RNA agent has a sequence sufficient to trigger the translational inhibition of the target RNA by the RNAi machinery or process. A RNA agent having a “sequence sufficiently complementary to a target RNA encoded by the target DNA sequence such that the target DNA sequence is chromatically silenced” means that the RNA agent has a sequence sufficient to induce transcriptional gene silencing, e.g., to down-modulate gene expression at or near the target DNA sequence, e.g., by inducing chromatin structural changes at or near the target DNA sequence.

The term “RNA” or “RNA molecule” or “ribonucleic acid molecule” refers to a polymer of ribonucleotides. The term “DNA” or “DNA molecule” or deoxyribonucleic acid molecule” refers to a polymer of deoxyribonucleotides. DNA and RNA can be synthesized naturally (e.g., by DNA replication or transcription of DNA, respectively). RNA can be post-transcriptionally modified. DNA and RNA can also be chemically synthesized. DNA and RNA can be single-stranded (i.e., ssRNA and ssDNA, respectively) or multi-stranded (e.g., double-stranded, i.e., dsRNA and dsDNA, respectively).

The term RNA includes noncoding (“ncRNAs”) and coding RNAs (i.e., mRNAs, as defined herein). ncRNAs are single- or double-stranded RNAs that do not specify the amino acid sequence of polypeptides (i.e., do not encode polypeptides). By contrast, ncRNAs affect processes including, but not limited to, transcription, gene silencing, replication, RNA processing, RNA modification, RNA stability, mRNA translation, protein stability, and/or protein translation. ncRNAs include, but are not limited to, bacterial small RNAs (“sRNA”), microRNAs (“miRNAs”), small temporal RNAs (“stRNAs”), and/or interspersed element RNAs (IRE RNAs).

The term “mRNA” or “messenger RNA” refers to a single-stranded RNA that specifies the amino acid sequence of one or more polypeptide chains. This information is translated during protein synthesis when ribosomes bind to the mRNA.

The term “transcript” refers to a RNA molecule transcribed from a DNA or RNA template by a RNA polymerase template. The term “transcript” includes RNAs that encode polypeptides (i.e., mRNAs) as well as noncoding RNAs (“ncRNAs”).

As used herein, the term “small interfering RNA” (“siRNA”) (also referred to in the art as “short interfering RNAs”) refers to an RNA agent, preferably a double-stranded agent, of about 10-50 nucleotides in length (the term “nucleotides” including nucleotide analogs), preferably between about 15-25 nucleotides in length, more preferably about 17, 18, 19, 20, 21, 22, 23, 24, or 25 nucleotides in length, the strands optionally having overhanging ends comprising, for example 1, 2 or 3 overhanging nucleotides (or nucleotide analogs), which is capable of directing or mediating RNA interference. Naturally-occurring siRNAs are generated from longer dsRNA molecules (e.g., >25 nucleotides in length) by a cell's RNAi machinery (e.g., the RISC complex).

As used herein, the term “miRNA” or “microRNA” refers to an RNA agent, preferably a single-stranded agent, of about 10-50 nucleotides in length (the term “nucleotides” including nucleotide analogs), preferably between about 15-25 nucleotides in length, more preferably about 17, 18, 19, 20, 21, 22, 23, 24, or 25 nucleotides in length, which is capable of directing or mediating RNA interference. Naturally-occurring miRNAs are generated from stem-loop precursor RNAs (i.e., pre-miRNAs) by Dicer.

As used herein, the term “pre-miRNA” refers to intermediate RNA precursors of miRNAs, e.g., stem-loop precursor RNAs cleaved by Dicer. The term “Dicer” as used herein, includes Dicer as well as any Dicer orthologue or homologue capable of processing dsRNA structures into siRNAs, miRNAs, siRNA-like or miRNA-like molecules.

Naturally occurring pre-miRNAs are generated from longer primary transcripts (pri-miRNAs) by a ribonuclease, e.g., Drosha. As used herein, the term “pri-miRNA” refers to RNA precursors of pre-miRNAs, e.g., RNA precursors which contain miRNAs and are cleaved by Drosha. The term “Drosha” as used herein, includes Drosha as well as any Drosha orthologue or homologue capable of processing dsRNA structures into pre-miRNAs or pre-miRNA-like molecules.

The term microRNA (or “miRNA”) is used interchangeably with the term “small temporal RNA” (or “stRNA”) based on the fact that naturally-occurring microRNAs (or “miRNAs”) have been found to be expressed in a temporal fashion (e.g., during development).

The term “shRNA” or “short hairpin RNA”, as used herein, refers to an RNA agent having a stem-loop structure, comprising a first and second region of complementary sequence, the degree of complementarity and orientation of the regions being sufficient such that base pairing occurs between the regions, the first and second regions being joined by a loop region, the loop resulting from a lack of base pairing between nucleotides (or nucleotide analogs) within the loop region.

The term “posttranscriptional gene silencing”, as used herein, refers to the silencing of a gene through a mechanism acting at a step subsequent to RNA transcription from the gene, e.g., an siRNA- or miRNA-like molecule may induce transcriptional gene silencing by inducing degradation of target RNA sequences or by inhibiting translation of target RNA sequences.

The term “transcriptional gene silencing”, as used herein, refers to the silencing of a gene through a mechanism acting at a step prior to RNA transcription from the gene, e.g., an siRNA- or miRNA-like molecule may induce transcriptional gene silencing by inducing chromatin silencing, e.g., heterochromatic silencing, of the gene. “Chromatin silencing”, as used herein, refers to a down modulation of gene expression effected through changes in chromatin structure, e.g., modification of chromatin components, such as histones.

The term “interspersed repetitive element” or “IRE” as used herein refers to a repetitive element in genomic DNA that is interspersed throughout the genome, e.g., transposable elements, mobile elements, retrotransposable elements, and the like. Preferred IREs of the invention include, but are not limited to, small interspersed elements (SINEs), long interspersed elements (LINEs), and LTR-retrotransposons.

The term “short interspersed element” or “SINE”, as used herein, refers to short (less than about 500 nucleotides in length) repetitive DNA sequences that are interspersed, e.g., not tandemly arrayed, throughout the genome. The term “Alu SINE” or “Alu element” refers to SINEs of the Alu family.

The term “long interspersed element” or “LINE”, as used herein, refers to long (greater than about 500 nucleotides in length) repetitive DNA sequences that are interspersed, e.g., not tandemly arrayed, throughout the genome.

The term “Alu RNA” refers to small (˜300 nucleotides in length) structured, noncoding RNA produced from Alu SINEs. The predicted structure of Alu RNA comprises two monomers, e.g., left and right monomers, at least one of which comprises a stem loop structure, e.g., hairpin structure (see Rubin, C. M. et al. 2002 Nuc. Acids Res. 30:3253-3261, the entire content of which is incorporated herein by reference).

The term “gene comprising an interspersed repetitive element (IRE)” refers to a gene having an IRE sequence or portion or derivative thereof, e.g., an intron or exon comprising an IRE sequence or portion or derivative thereof. A gene comprising an interspersed repetitive element is preferably a gene in which an exon (e.g., alternatively spliced exon) comprises an IRE sequence or portion or derivative thereof, e.g., Alu exon.

The term “nucleoside” refers to a molecule having a purine or pyrimidine base covalently linked to a ribose or deoxyribose sugar. Exemplary nucleosides include adenosine, guanosine, cytidine, uridine and thymidine. The term “nucleotide” refers to a nucleoside having one or more phosphate groups joined in ester linkages to the sugar moiety. Exemplary nucleotides include nucleoside monophosphates, diphosphates and triphosphates. The terms “polynucleotide” and “nucleic acid molecule” are used interchangeably herein and refer to a polymer of nucleotides joined together by a phosphodiester linkage between 5′ and 3′ carbon atoms.

The term “nucleotide analog” or “altered nucleotide” or “modified nucleotide” refers to a non-standard nucleotide, including non-naturally occurring ribonucleotides or deoxyribonucleotides. Preferred nucleotide analogs are modified at any position so as to alter certain chemical properties of the nucleotide yet retain the ability of the nucleotide analog to perform its intended function. Examples of positions of the nucleotide which may be derivatized include the 5 position, e.g., 5-(2-amino)propyl uridine, 5-bromo uridine, 5-propyne uridine, 5-propenyl uridine, etc.; the 6 position, e.g., 6-(2-amino)propyl uridine; the 8-position for adenosine and/or guanosines, e.g., 8-bromo guanosine, 8-chloro guanosine, 8-fluoroguanosine, etc. Nucleotide analogs also include deaza nucleotides, e.g., 7-deaza-adenosine; O— and N-modified (e.g., alkylated, e.g., N6-methyl adenosine, or as otherwise known in the art) nucleotides; and other heterocyclically modified nucleotide analogs such as those described in Herdewijn, Antisense Nucleic Acid Drug Dev., August 2000 10(4):297-310.

Nucleotide analogs may also comprise modifications to the sugar portion of the nucleotides. For example the 2′ OH-group may be replaced by a group selected from H, OR, R, F, Cl, Br, I, SH, SR, NH₂, NHR, NR₂, COOR, or OR, wherein R is substituted or unsubstituted C₁-C₆ alkyl, alkenyl, alkynyl, aryl, etc. Other possible modifications include those described in U.S. Pat. Nos. 5,858,988, and 6,291,438.

The phosphate group of the nucleotide may also be modified, e.g., by substituting one or more of the oxygens of the phosphate group with sulfur (e.g., phosphorothioates), or by making other substitutions which allow the nucleotide to perform its intended function such as described in, for example, Eckstein, Antisense Nucleic Acid Drug Dev. April 2000 10(2):117-21, Rusckowski et al. Antisense Nucleic Acid Drug Dev. October 2000 10(5):333-45, Stein, Antisense Nucleic Acid Drug Dev. October 2001 11(5): 317-25, Vorobjev et al. Antisense Nucleic Acid Drug Dev. April 2001 11(2):77-85, and U.S. Pat. No. 5,684,143. Certain of the above-referenced modifications (e.g., phosphate group modifications) preferably decrease the rate of hydrolysis of, for example, polynucleotides comprising said analogs in vivo or in vitro.

The term “oligonucleotide” refers to a short polymer of nucleotides and/or nucleotide analogs. The term “RNA analog” refers to an polynucleotide (e.g., a chemically synthesized polynucleotide) having at least one altered or modified nucleotide as compared to a corresponding unaltered or unmodified RNA but retaining the same or similar nature or function as the corresponding unaltered or unmodified RNA. As discussed above, the oligonucleotides may be linked with linkages which result in a lower rate of hydrolysis of the RNA analog as compared to an RNA molecule with phosphodiester linkages. For example, the nucleotides of the analog may comprise methylenediol, ethylene diol, oxymethylthio, oxyethylthio, oxycarbonyloxy, phosphorodiamidate, phophoroamidate, and/or phosphorothioate linkages. Preferred RNA analogues include sugar- and/or backbone-modified ribonucleotides and/or deoxyribonucleotides. Such alterations or modifications can further include addition of non-nucleotide material, such as to the end(s) of the RNA or internally (at one or more nucleotides of the RNA). An RNA analog need only be sufficiently similar to natural RNA that it has the ability to mediate (mediates) RNA interference.

As used herein, the term “isolated RNA” (e.g., “isolated SINE RNA”, “isolated Alu RNA” or “isolated RNAi agent”) refers to RNA molecules which are substantially free of other cellular material, or culture medium when produced by recombinant techniques, or substantially free of chemical precursors or other chemicals when chemically synthesized.

The term “in vitro” has its art recognized meaning, e.g., involving purified reagents or extracts, e.g., cell extracts. The term “in vivo” also has its art recognized meaning, e.g., involving living cells, e.g., immortalized cells, primary cells, cell lines, and/or cells in an organism.

As used herein, the term “druggable target” refers to a target (i.e., gene or gene product) having certain desired properties which indicate a potential for drug discovery, i.e., for use in the identification, research and/or development of therapeutically relevant compounds. A druggable target is distinguished based on certain physical and/or functional properties selected by a person skilled in the art of drug discovery. A druggable target (i.e., gene or gene product) of the instant invention, for example, is distinguished from other genes and/or gene products based on the fact that that it is regulated by RNAi, preferably by RNAi mediated via an IRE RNA, e.g., SINE RNA, Alu RNA, or derivative thereof.

Based on the fact that these targets may be regulated by RNAi, it is believed that the targets are important in essential cellular processes, for example, maintenance of cellular homeostasis, host cell defense mechanisms, and the like. Control of such processes, including situations in which such processes are misregulated (i.e., in the biology of a disease), has obvious therapeutic appeal. Additional criteria for identifying and/or selecting druggable targets include, but are not limited to (1) cellular localization susceptible to systemically administered (e.g., orally administered) drugs; (2) homology or similarity to other genes and/or gene products (e.g., member of a gene family) previously successfully targeted; and (3) data (e.g., expression and/or activity data) indicating a role for the gene/gene product at a critical intervention points in a disease pathway.

The term “antiviral drug target”, as used herein, refers to a target (i.e., gene or gene product) having certain desired properties which indicate a potential for antiviral drug discovery, i.e., for use in the identification, research and/or development of compounds useful in antiviral therapies. A druggable target (i.e., gene or gene product) of the instant invention, for example, is indicated as a druggable target based on the fact that endogenous RNAs, in particular, IRE RNAs, e.g., SINE RNAs, Alu RNAs, or derivatives thereof can act as mediators (e.g., substrates and/or inhibitors) of RNAi.

A gene “involved” in a disorder includes a gene, the normal or aberrant expression or function of which effects or causes a disease or disorder or at least one symptom of said disease or disorder

The phrase “examining the function of a gene in a cell or organism” refers to examining or studying the expression, activity, function or phenotype arising there from.

Various methodologies of the instant invention include a step that involves comparing a value, level, feature, characteristic, property, etc. to a “suitable control”, referred to interchangeably herein as an “appropriate control”. A “suitable control” or “appropriate control” is any control or standard familiar to one of ordinary skill in the art useful for comparison purposes. In one embodiment, a “suitable control” or “appropriate control” is a value, level, feature, characteristic, property, etc. determined prior to performing an RNAi methodology, as described herein. For example, a transcription rate, mRNA level, translation rate, protein level, biological activity, cellular characteristic or property, genotype, phenotype, etc. can be determined prior to introducing an RNAi agent of the invention into a cell or organism. In another embodiment, a “suitable control” or “appropriate control” is a value, level, feature, characteristic, property, etc. determined in a cell or organism, e.g., a control or normal cell or organism, exhibiting, for example, normal traits. In yet another embodiment, a “suitable control” or “appropriate control” is a predefined value, level, feature, characteristic, property, etc.

II. Interspersed Repetitive Elements (IREs)

All eukaryotic genomes contain DNA sequences, termed “repetitive elements”, which are present in multiple copies throughout the genome. These repetitive sequences can be tandemly arrayed, as, for example, in the case of micro satellite, minisatellite and telomeric DNA. Alternatively, repetitive elements can be interspersed throughout the genome, such as, for example, mobile elements and processed pseudogenes. Interspersed elements can be subdivided on the basis of size, with short interspersed elements (SINEs) being less than 500 bp long, and the remainder of interspersed elements considered to be long interspersed elements (LINEs). LTR-retrotransposons are also considered repetitive interspersed elements. Mobile elements are highly abundant, constituting over 45% of the human genome. These elements use extensive cellular resources in their replication, expression and amplification, and, as a result of negative effects of their transposition, contribute to a notable number of human diseases. It remains a topic of debate whether mobile elements are primarily an intracellular plague that attacks the host genome and exploits cellular resources, or whether they are tolerated because of their occasional positive influence in genome evolution.

IRE RNA sequences have been extensively described and are known to one of skill in the art. For example, an assembly and annotation of the first draft sequence of the entire human genome that includes a comprehensive analysis of repeated DNA sequences can be found in “International Human Genome Sequencing Consortium: Initial sequencing and analysis of the human genome” (2001 Nature 409:860-921), the entire contents of which are incorporated herein by reference. Characteristics of repetitive sequences can also be found in “Densities, length proportions, and other distributional features of repetitive sequences in the human genome estimated from 430 megabases of genomic sequences” (Z. Gu et al., 2000 Gene 259:81-88), the entire contents of which are incorporated herein by reference. A compilation of mobile elements which have been found to functionally significant in the genome can be found in R. J. Britten et al. (“Mobile elements inserted in the distant past have taken on important functions” 1997 Gene 205: 177-182), the entire contents of which are incorporated herein by reference.

IRE RNA sequences, e.g., Alu RNA sequences, can be identified using tools well known to one of skill in the art. For example, computational tools have been developed for systematic genome annotation of repeat families. One example of a computation tool that can be used to identify IRE sequences, e.g., Alu RNA sequences, is the widely used program RepeatMasker (A. F. A. Smit and P. Green), which uses precompiled representative sequence libraries to find homologous copies of known repeat families. RepeatMasker is indispensable in genomes in which repeat families have already been analyzed. Another computational tool that can be used to identify IRE sequences, e.g., Alu RNA sequences, is a novel automated approach developed for de novo repeat identification referred to as the RECON algorithm, as described in Bao and Eddy (2002 Genome Research 12:1269-1276), the entire contents of which are incorporated herein by reference. This approach uses multiple alignment information to infer element boundaries and biologically reasonable clustering of sequence families. The algorithm has been implemented as RECON, a set of C programs, and Perl scripts. The RECON package, including a demo and more materials, is available and can be found at the following World Wide Web site: genetics.wustl.edu/eddy/recon.

A. LTR Retrotransposons

LTR retrotransposons are autonomous elements in that, although they are dependent on many cellular proteins for their amplification cycle, they do encode one or more of the necessary activities within the element. LTR retrotransposons are similar to retroviruses in structure, with transcriptional regulatory sequences located in the flanking LTRs, a priming site to allow priming of the reverse transcription usually located downstream of the first LTR, and several open reading frames encoding proteins necessary for retrotranspositions. These proteins include domains for an endonuclease for cleaving the genomic integration site and reverse transcriptase to copy the RNA to DNA. Unlike retroviruses, however, LTR retrotransposons lack envelope genes and genomic components required for making a functional viral capsule. Nonautonomous versions of LTR retrotransposons also exist, in which the LTR structure and primer-binding site are maintained but some or all of the coding capacity is deleted.

B. SINES and LINES

Retrotransposons lacking the LTR repeat, e.g., non-LTR retrotransposons, can be subdivided into short interspersed elements (SINEs) and long interspersed elements (LINEs). SINEs are nonautonomous elements in that they also amplify through a process of retrotransposition, but require at least one activity that is supplied by an autonomous element for their retrotransposition. SINEs are small elements, usually 90-300 bp in length, which are transcribed by RNA polymerase III. These elements are ancestrally derived from various tRNA genes or the 7SL RNA gene. SINEs have no protein coding capacity, and evidence suggests that they are dependent on LINEs for their amplification (Okada and Hamada 1997; Weiner et al. 1986; Danils and Deininger 1986). The copy number of a single SINE can exceed 10⁶.

The most abundant SINEs in the human genome are “Alu elements” or “Alu SINEs”. Alu elements were originally identified as a family of repeats containing a recognition site for the restriction enzyme AluI (C. M. Houch et al., 1979 J. Mol. Biol. 132:289-306). The origins of these Alu elements that are dispersed throughout the human genome can be traced to an initial gene duplication early in primate evolution, and to the subsequent and continuing amplification of these elements. Today, Alu SINEs are estimated to be present in the human genome at over one million copies and to comprise more than 10% of the mass of the human genome (International Human Genome Sequencing Consortium 2001 Nature 409: 860-921). Alu insertions are estimated to account for ˜0.1% of all human genetic disorders, such as neurofibromatosis, hemophilia, breast cancer, Apert syndrome, cholinesterase deficiency and complement deficiency (P. L. Deininger and M. A. Batzer 1999 Mol. Genet. Metab. 67:183-193). In the human genome, Alu repeats are most commonly found in gene-rich chromosomal regions, and specifically in untranslated regions including introns, 3′ untranslated regions of genes and intergenic genomic regions. Alu repetitive elements are transcribed by RNA polymerase III to produce non-translated RNA transcripts.

The origin and amplification of Alu elements are evolutionarily recent events that coincided with the radiation of primates in the past 65 million years. Detailed sequence analysis of the structure of Alu element RNAs has indicated that Alu elements were ancestrally derived from the 7SL RNA gene, which forms part of the ribosome complex. Therefore, the origins of more than 1.1 million Alu elements that are dispersed throughout the human genome can be traced to an initial gene duplication early in primate evolution, and to the subsequent and continuing amplification of these elements. This type of duplication, followed by the expansion of a SINE family, has occurred sporadically throughout evolutionary history in mammalian and non-mammalian genomes. The origins of a variety of SINEs can be traced to the genes of various small, highly structured RNAs, such as transfer RNA genes, the transcription of which depends on RNA polymerase III (REFS 1,15-18). The expansion of SINEs of different origins has occurred simultaneously in several diverse genomes, and although the reasons for this simultaneous expansion are unknown, there have been many interesting discussions about the factors that might have contributed to it.

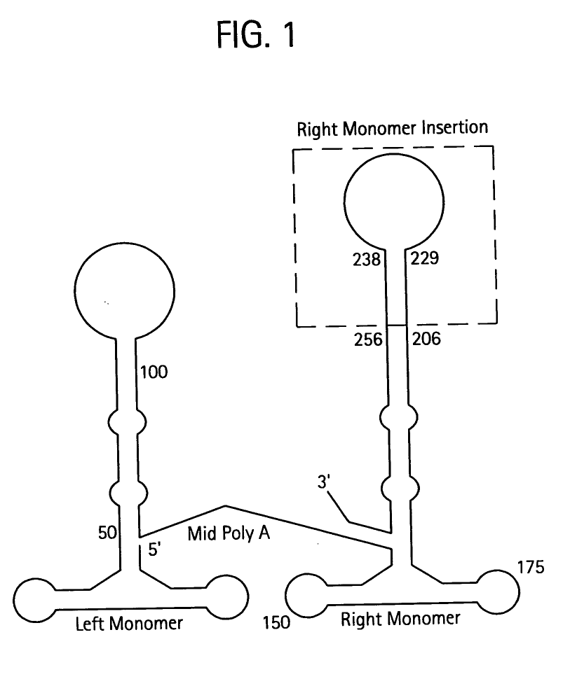

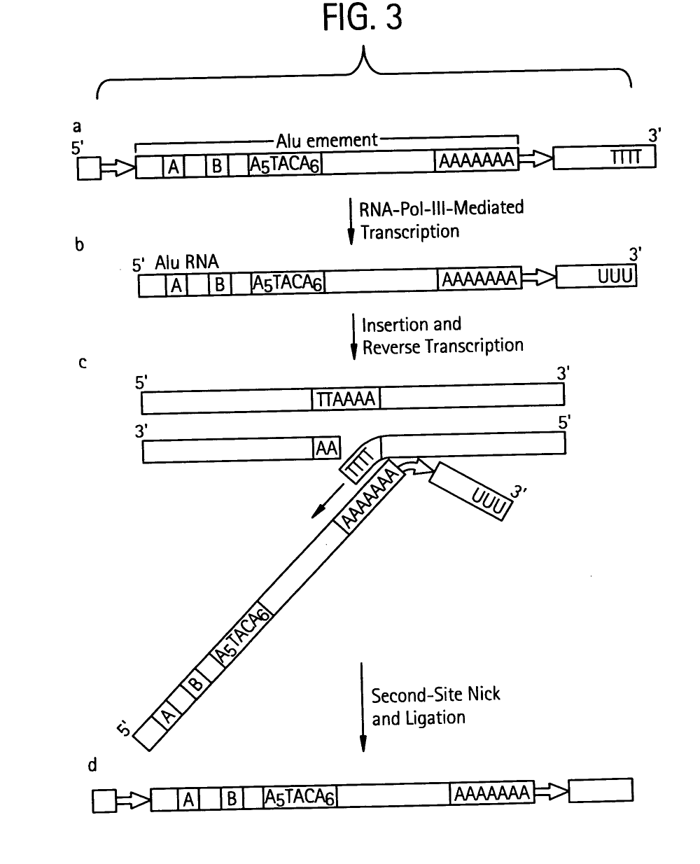

Alu RNA sequences have been extensively described and are known to one of skill in the art. For example, an extensive description of Alu repeat RNA sequences can be found in “Alu Repeats and Human Genomic Diversity” (Batzer and Deininger 2002 Nature Reviews: Genetics 3:370-380), the entire contents of which are incorporated herein by reference. Alu RNA repetitive sequences can be identified by one skilled in the art on the basis of their structure and/or consensus sequences. The typical structure of an Alu element is shown in FIG. 3A. The structure of each Alu element is bi-partite, with the 3′ half containing an additional 31-bp insertion relative to the 5′ half. Full-length Alu RNA transcripts are ˜300 bp long (depending on the length of the 3′ oligo(dA)-rich tail). The elements also contain a central A-rich region (A₅TACA₆) and are flanked by short intact direct repeats that are derived from the site of insertion. The 5′ half of each sequence contains an RNA-polymerse-III promoter. The 3′-terminus of the Alu element almost always consists of a run of As that is only occasionally interspersed with other bases. As further depicted in FIG. 3, Alu elements increase in number by retrotransposition, a process that involves reverse transcription of an Alu-derived RNA polymerase III transcript. As the Alu element does not code for an RNA-polymerase-III termination signal, its transcript will therefore extend into the flanking unique sequence (FIG. 3B). The typical RNA-polymerase-III terminator signal is a run of four or more Ts on the sense strand, which results in three Us at the 3′ terminus of most transcripts. It has been proposed that the run of As at the 3′ end of the Alu might anneal directly at the site of integration in the genome for target-primed reverse transcription (mauve arrow indicates reverse transcription) (FIG. 3C). It seems likely that the first nick at the site of insertion is often made by the L1 endonuclease at the TTAAAA consensus site. The mechanism for making the second-site nick on the other strand and integrating the other end of the Alu element remains unclear. A new set of direct repeats (red arrows) is created during the insertion of the new Alu element (FIG. 3D). Importantly, full length Alu RNAs, as depicted in FIG. 1, have a distinct predicted secondary structure comprising a left and right monomer, each of which contains a hairpin structure (C. M. Rubin et al. 2002 Nuc. Acids Res. 30:3253-3261). The secondary structure of Alu RNAs and, specifically, the hairpins of the left and right monomers, are highly similar to the stem-loop structure of endogenous cellular microRNA (miRNA) precursors.

The consensus sequences of ALU repeat sequences are well described. The human Alu family is composed of several distinct subfamilies of different genetic ages that are characterized by a hierarchical series of mutations. The first report of subfamily structure in Alu elements was described by Slagel et al. (1987 Mol. Biol. Evol. 4:19-29, the entire contents of which are incorporated herein by reference). A number of human Alu elements that share common diagnostic sequence features and comprise subfamilies or clades that have expanded in different evolutionary time frames have been identified and described (Deininger and Batzer 1993 Evol. Biol. 27:157-196, the entire contents of which are incorporated herein by reference). The consensus Alu sequence contains nine potential 5′ splice sites (donor sites) and fourteen 3′ splice sites (acceptor sites) (Sorek et al 2002 Genome Res. 12:1060-1067). However, these splice sites are not evenly distributed throughout the Alu element. Only four of the potential splice sites reside on the plus strand of the Alu element, whereas the minus strand contains nineteen. Thus it is much more likely that intronic Alu elements can be converted into exons when their orientation opposes the direction of transcription of the host gene.

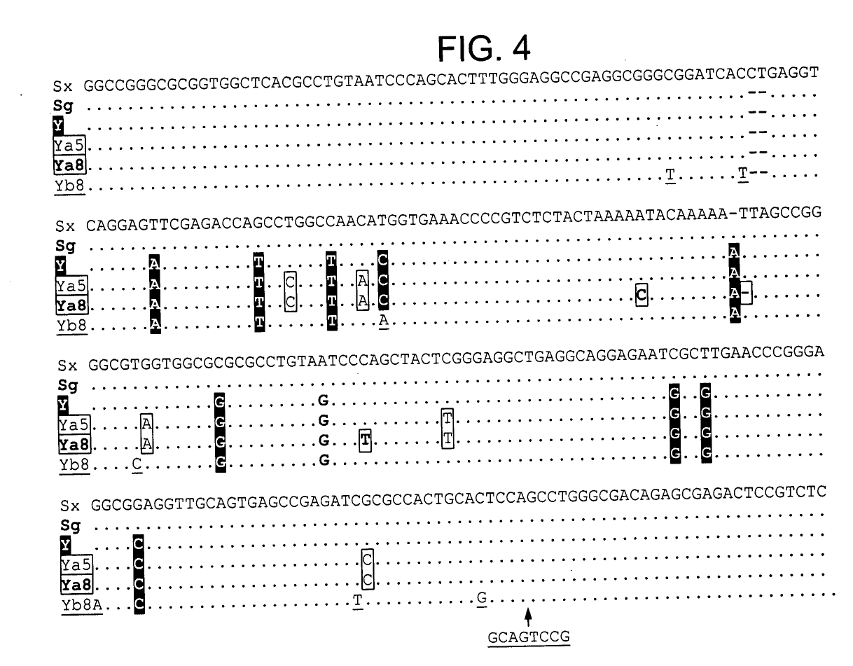

There are several subfamilies of Alu sequences, the most prevalent of which are the J and S subfamilies (“A fundamental division in the Alu family of repeated sequences” Jurka and Smith 1988 Proc. Natl. Acad. Sci. U.S.A. 85:4775-4778, the entire contents of which are incorporated herein by reference). The consensus sequences of several Alu subfamilies are depicted in FIG. 4. In FIG. 4, the consensus sequence for the Alu Sx subfamily is shown at the top (SEQ ID NO:1), with the sequences of progressively younger Alu subfamilies underneath. The dots represent the same nucleotides as the consensus sequence. Deletions are shown as dashes, and mutations are shown in shaded boxes. Each of the newer subfamilies, such as Ya5 or Yb8, has all the mutations of the ancestral Alu elements, as well as five or eight extra mutations, respectively, that are diagnostic for the particular Alu subfamily. This figure primarily illustrates the newer subfamilies and does not show many of the older Alu subfamilies. Older Alu subfamilies are characterized by the smallest number of diagnostic subfamily-specific mutations. These older elements have also accumulated the largest number of random mutations (up to 20% pair-wise divergence), which confirms their ancient origin. By contrast, the younger families of Alu elements are characterized by an increasing number of subfamily-specific mutations, together with a smaller number of random mutations (as little as 0.1% pair-wise divergence) that accumulate after the individual Alu elements integrate into the genome.

Despite the remarkable abundance of Alu repetitive elements in eukaryotic genomes, their functions and/or effects remain largely unknown. One potential clue to Alu RNA function lies in the observation that Alu RNA expression increases in response to cellular stress, to viral infection and to translational inhibition (T. Li and C. W. Schmid 1993 Gene 276: 135-141;W. M. Liu et al., 1995 Nuc. Acids Res. 23:1758-1765). Alu RNA can bind the cellular protein kinase, PKR, a key component of the innate mammalian immune response (C. M. Rubin et al. 2002 Nuc. Acids Res. 30:3253-3261; MB Matthews and T. Shenk 1991 J. Virol. 65(11):5657-62). In addition, Alu RNAs have been observed to stimulate the translational expression of exogenous reporter genes (Rubin et al. (2002) Nuc Acids Res. 30 (14): 3253-3261); this stimulation does not affect the rate of global protein synthesis or mRNA expression or stability. This latter finding indicates that Alu RNAs may play a role in maintaining or regulating translation. Intriguingly, it has been found in C. elegans and Drosophila melanogaster that mutation of components of the RNAi pathway increases the mobilization of genetic elements (R. F. Ketting et al. 1999 Cell 99:133-141; R. W. Carthew 2001 Curr. Opin. Cell Biol. 13(2):244-248).

Long interspersed elements (LINEs) are larger than SINEs, e.g., usually greater than 500 bp in length, and are also transcribed by RNA polymerase III. Evidence from insect and mammalian species indicates that LINEs are able to transpose autonomously. LINEs share two features with SINEs, their 3′ A stretch and direct repeats of variable length. The most important LINE is L1, an element that is currently actively amplifying and, together with Alu elements, make up about 25% of the genome.

Based on the structural similarity between, at least, Alu SINE RNA and miRNA precursors (e.g., pri-miRNAs and pre-miRNAs), the instant inventors propose that interspersed repetitive element (IRE) RNAs, e.g., SINE, LINE or LTR-retrotransposon RNAs, are incorporated into the RNAi pathway. For example, Alu RNAs may be initially processed by Drosha and subsequently processed by the enzyme Dicer, thereby producing functional siRNAs or miRNAs to regulate gene expression during times of cellular insult. Alternatively, the IRE RNAs are proposed to act as competitive inhibitors for the components of the RNAi pathway, effectively preventing its normal processing and gene regulation. IRE loci may also be used as a template for the construction of gene therapy vectors or viruses to produce functional processed siRNAs or miRNAs. Finally, the involvement of Alu RNA in the RNAi pathway may provide a mechanistic explanation for the observed phenomenon of Alu RNAs' effect on exogenous gene expression.

The sequences of IRE RNA, e.g., Alu repeats, can be found, for example, in databases known to those of ordinary skill in the art, e.g., Alu repeat databases of the National Center for Biotechnology Information (NCBI), INFOBIOGEN, and EMBL Outstation, European Bioinformatics Institute. These IRE RNA sequences (and derivatives thereof), e.g., Alu RNA sequences, have utility as substrates and/or inhibitors as described herein. Corresponding IRE DNA sequences (e.g., having utility, either in their entirety or in part, as vector sequences) can be found in the EMBL Nucleotide Sequence Database using the Accession Nos. set forth in the databases.

III. miRNAs, siRNAs, miRNA-Like and siRNA-Like Molecules

MicroRNAs (miRNAs) are small (e.g., 19-25 nucleotides), single-stranded noncoding RNAs that are processed from ˜70 nucleotide hairpin precursor RNAs by Dicer. siRNAs are of a similar size and are also non-coding, however, siRNAs are processed from long dsRNAs and are usually double stranded (e.g., endogenous siRNAs). miRNAs can pair with target mRNAs that contain sequences only partially complementary (e.g., 50%, 60%, 70%, 80%) to the miRNA. Such pairing results in repression of mRNA translation without altering mRNA stability. Recently, it has also been demonstrated that miRNAs are capable of mediating RNAi (Hutvagner and Zamore (2002) Science 297:2056-2060). As expression of the precursor RNAs (i.e., pri-miRNAs and pre-miRNAs) is often developmentally regulated, miRNAs are often referred to interchangeably in the art as “small temporal RNAs” or “stRNAs”.

C. elegans contains approximately 100 endogenous miRNA genes, about 30% of which are conserved in vertebrates. The present inventors propose that certain IRE RNAs (e.g., Alu RNAs) can be processed by Drosha and/or Dicer (or a homologue or orthologue thereof) into small RNAs capable of mediating RNAi. Accordingly, such IRE RNA-derived small RNAs are referred to herein as miRNA like (in instances where the active RNA is single stranded) or siRNA-like (in instances where the active RNA is double stranded).

IV. Experimental Applications

As described herein, IRE RNAs (e.g., Alu RNAs) have utility as substrates and/or inhibitors of RNAi. Moreover, the present invention provides methods for identifying the targets of IRE RNAs (e.g., Alu RNAs). IRE RNAs (e.g., Alu RNAs) (and/or RNA agents derived therefrom) as well as IRE RNA targets can further be used experimentally, for example, in creating knockout and/or knockdown cells or organisms, in functional genomics and/or proteomics applications, in screening assays, and the like.

A. Screening Assays

In one aspect of the invention, IRE RNAs (e.g., Alu RNAs) (and/or RNA agents derived therefrom) as well as IRE RNA targets, as identified herein, are suitable for use in methods to identify and/or characterize potential pharmacological agents, e.g. identifying new pharmacological agents from a collection of test substances and/or characterizing mechanisms of action and/or side effects of known pharmacological agents.

1. IRE RNAs as Substrates of RNAi

IRE RNAs (e.g., Alu RNAs) may function as substrates for the RNAi pathway and become processed to produce siRNA or miRNA-like molecules that may function to control viral and/or host cell gene expression. Accordingly, in one embodiment, the invention features a system for identifying and/or characterizing pharmacological agents acting on, for example, an IRE RNA:target RNA pair comprising: (a) a cell capable of expressing the target RNA, (b) at least one IRE RNA molecule (or RNA agent derived therefrom) capable of modulating (e.g., inhibiting) the expression of said target RNA, and (c) a test substance or a collection of test substances wherein pharmacological properties of said test substance or said collection are to be identified and/or characterized. In another embodiment, the invention features a system for identifying and/or characterizing pharmacological agents acting on, for example, a IRE RNA:target RNA pair comprising: (a) an organism (e.g., a non-human eukaryotic organism) capable of expressing the target RNA, (b) at least one IRE RNA molecule (or RNA agent derived therefrom) capable of modulating (e.g., inhibiting) the expression of said target RNA, and (c) a test substance or a collection of test substances wherein pharmacological properties of said test substance or said collection are to be identified and/or characterized.

Preferred cells for use in the screening assays of the invention are eukaryotic cells, although screening in prokaryotic cells is also contemplated. In one embodiment, the cell is a plant cell. In another embodiment, the cell is an insect cell. In yet another embodiment, the cell is a mammalian cell (e.g., a human or murine cell). In yet another embodiment, the cell is an avian cell. Preferred organisms for use in the screening assays of the invention include lower organisms, for example, C. elegans. Test substances are contacted with the cell or organism capable of expressing the target RNA (i.e., the test cell or organism, respectively) before, after or simultaneously with the IRE RNA agent.

Cells or organisms are assayed, for example, for an indicator of RNAi. As used herein, the phrase “indicator of RNAi” refers to any detectable marker, readout, etc. which is indicative of RNAi activity or an RNAi process occurring in said cell or organism. Levels of substrates or products of an RNAi process are preferred indicators. For example, in instances where a IRE RNA is a substrate for an RNAi process, levels (e.g., decreasing levels) of IRE RNA are indicative of RNAi. Alternatively, levels (e.g., increasing levels) of miRNA- or siRNA-like molecules are indicative of siRNA-like molecules. In another embodiment, levels of intermediate products (e.g., small duplex RNA are indicative of RNAi. Other preferred indicators include levels of target RNA (e.g., target mRNA) and/or levels of protein encoded by a target mRNA. The latter, for example, can be indicative of target cleavage (i.e., a siRNA or miRNA-like function) and/or translational repression (i.e., a mi-RNA-like function). In certain embodiments, one or more substrate, product, intermediate, etc. is labeled (e.g., enzymatically, fluorescently or radioisotypically labeled to facilitate detection). Enzymatically labeled reagents are often assayed in the presence of a variety of colorimetric substances. Indirect assays, for example, reporter gene assays sensitive to levels of proteins encoded by target mRNAs, are also suitable as indicators of RNAi. In preferred embodiments, a system as described above can further comprise suitable controls.

The test compounds of the present invention can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Drug Des. 12:145). The test compounds of the present invention can be obtained using nucleic acid libraries, e.g., complementary DNA libraries (see S. Y. Sing (2003) Methods Mol Biol 221:1-12), DNA or RNA aptamer libraries (see C. K. O'Sullivan 2002 Anal Bioanal Chem 372(1):44-48; J. J. Toulme 2000 Curr Opin Mol Ther 2(3):318-24; J. J. Toulme et al., 2001 Prog Nucleic Acid Res Mol Biol 69:1-46) and by using in vitro evolution approaches, e.g., in vitro evolution of nucleic acids (see, e.g., J. A. Bittker et al. 2002 Curr Opin Chem Biol 6(3):367-374).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner USP '409), plasmids (Cull et al. (1992) Proc Natl Acad Sci USA 89:1865-1869) or on phage (Scott and Smith (1990) Science 249:386-390); (Devlin (1990) Science 249:404-406); (Cwirla et al. (1990) Proc. Natl. Acad. Sci. 87:6378-6382); (Felici (1991) J. Mol. Biol. 222:301-310); (Ladner supra.)).

In a preferred embodiment, the library is a natural product library, e.g., a library produced by a bacterial, fungal, or yeast culture. In another preferred embodiment, the library is a synthetic compound library.

Compounds or agents identified according to such screening assays can be used therapeutically or prophylactically either alone or in combination, for example, with an Alu RNA (or derivative thereof) of the invention, as described supra.

In another embodiment of the invention, a system is featured for identifying and/or characterizing a druggable target, for example, a cellular or viral gene, comprising: (a) an assay composition comprising an RNAi pathway molecule and a IRE RNA (e.g., Alu RNA); (b) assaying for expression of a candidate RNA, wherein a change in expression of the candidate RNA indicates that a gene or protein corresponding to the RNA is a druggable target. In a related embodiment, the invention features a system for identifying and/or characterizing a druggable target, for example, a cellular or viral gene, comprising: (a) a cell or organism comprising an RNAi pathway molecule and a IRE RNA (e.g., Alu RNA), (b) assaying for expression of a candidate RNA, wherein a change in expression of the candidate RNA indicates that a gene or protein corresponding to the RNA is a druggable target.

Candidate target RNAs of IRE RNAs can be identified by using methodologies commonly known to the skilled artisan. For example, computer algorithms can be used to search a host genome for sequences of homology to a IRE RNA sequence. Preferably, an IRE RNA sequence having homology to a host gene is located within a duplex, e.g., stem region, of the IRE RNA. In preferred embodiments of this approach to identifying target RNAs of IRE RNAs, genome sequences are searched for sequences having at least about 50%, 60%, 70%, 80%, 90% or 100% homology to the IRE RNA sequence. Another approach to identify candidate target RNAs of IRE RNAs is the use of solid-based nucleic acid arrays, e.g., DNA and/or RNA arrays or “chips”, to identify genes whose expression is changed upon IRE RNA expression, e.g., upon viral infection, in a cell or organism. Solid-based nucleic acid array technologies are well known to those skilled in the relevant art. The IRE RNA can be expressed in the cell or organism from e.g., a virus, viral-derived vector, plasmid, transgene, and the like. In this approach, gene expression in the presence of IRE RNA expression can be measured and compared, for example, to gene expression in the absence of IRE RNA expression or to gene expression in the presence of an IRE RNA that has been modified so that the siRNA- or miRNA-like molecule generated from the IRE RNA is inactivated. In cases where the IRE RNA is known or suspected to play a role in a particular function, e.g., a cellular or viral function, a subset of candidate target RNAs, e.g., cellular or viral RNAs, previously identified as being involved in that function can be selected and analyzed for changes in gene expression. In cases where the candidate target RNA is suspected to be a viral RNA, gene expression in the presence of IRE RNA expression can be measured and compared, for example, in a cell or organism deficient or lacking in PKR activity.

2. IRE RNAs as Inhibitors of RNAi

IRE RNAs (e.g., Alu RNAs) can function as inhibitors of the RNAi pathway, thereby modulating viral and/or host cell gene expression normally regulated by an RNAi-mediated function. For example, IRE RNAs may be incorporated into a Drosha, RISC or Dicer-containing complex and thereby compete with alternate substrates for the RNAi pathway.

Accordingly, in one aspect, the instant invention features a method for modulating RNAi, e.g., inhibiting RNAi, in a cell, comprising introducing into the cell an IRE RNA or modulatory, e.g., inhibitory, derivative thereof, such that RNAi in the cell is inhibited. In a related embodiment, the invention provides a method of inhibiting the incorporation of a siRNA or miRNA into a cellular Dicer or RISC complex, comprising introducing into the cell an isolated IRE RNA or inhibitory derivative thereof, such that incorporation of the siRNA or miRNA into the complex is inhibited.

In another aspect, the invention provides a method for identifying a therapeutic agent, comprising: (a) contacting a cell with a test agent, said cell comprising an RNAi pathway and an IRE RNA, wherein the ribonucleotide inhibits the RNAi pathway; and (b) detecting an indicator of the RNAi pathway, wherein an agent is identified based on its ability to modulate (e.g., promote) inhibition of the RNAi pathway.

In still another aspect, the invention features a method for identifying a therapeutic agent, comprising: (a) contacting an assay composition with a test agent, wherein said assay composition comprises a RNAi pathway molecule and a IRE RNA which inhibits the activity of said RNAi pathway molecule; and (b) detecting activity of said RNAi pathway molecule, wherein said agent is identified based on its ability to modulate (e.g., further inhibit) the inhibition of said RNAi pathway molecule. In a related embodiment, the invention further features a method for identifying a therapeutic agent, comprising: (a) contacting an assay composition with a test agent, wherein said assay composition comprises a IRE RNA and a RNAi pathway molecule capable of interacting with or altering the IRE RNA; and (b) detecting the ability of the RNAi pathway molecule to interact with or alter the IRE RNA, wherein said agent is identified based on its ability to modulate the interaction of the IRE RNA with RNAi pathway molecule or alteration of the IRE RNA by the RNAi pathway molecule.

B. Knockout and/or Knockdown Cells or Organisms

An IRE RNA (e.g., Alu RNA) (or derivative thereof) (either known or identified by the methodologies of the present invention) can be used in a functional analysis of the corresponding target RNA (either known or identified by the methodologies of the present invention). Such a functional analysis is typically carried out in eukaryotic cells, or eukaryotic non-human organisms, preferably mammalian cells or organisms and most preferably human cells, e.g. cell lines such as HeLa or 293 or rodents, e.g. rats and mice. By administering a suitable RNA agent, a specific knockout or knockdown phenotype can be obtained in a target cell, e.g. in cell culture or in a target organism. Alternatively, such a functional analysis can be carried out in prokaryotic organisms.

Thus, further subject matter of the invention includes cells (e.g., eukaryotic cells) or organisms (e.g., eukaryotic non-human organisms) exhibiting a target gene-specific knockout or knockdown phenotype resulting from a fully or at least partially deficient expression of at least one endogenous target gene wherein said cell or organism is transfected with or administered, respectively, at least one IRE RNA (e.g., Alu RNA) (or derivative thereof, e.g., inhibitory derivative) or vector comprising DNA encoding said IRE RNA capable of inhibiting the expression of the target gene. It should be noted that the present invention allows a target-specific knockout or knockdown of several different endogenous genes based on the specificity of the IRE RNA (e.g., Alu RNA) (or derivative thereof, e.g., inhibitory derivative) transfected or administered.

Gene-specific knockout or knockdown phenotypes of cells or non-human organisms, particularly of human cells or non-human mammals may be used in analytic to procedures, e.g. in the functional and/or phenotypical analysis of complex physiological processes such as analysis of gene expression profiles and/or proteomes. Preferably the analysis is carried out by high throughput methods using oligonucleotide based chips.

C. Functional Genomics and/or Proteomics

Another utility of the present invention could be a method of identifying gene function in an organism comprising the use of an IRE RNA (or derivative thereof, e.g., inhibitory derivative) to inhibit the activity of a target gene of previously unknown function. Instead of the time consuming and laborious isolation of mutants by traditional genetic screening, functional genomics would envision determining the function of uncharacterized genes by employing the invention to reduce the amount and/or alter the timing of target gene activity.

The ease with which RNA agents can be introduced into an intact cell/organism containing the target gene allows the present invention to be used in high throughput screening (HTS). Solutions containing an IRE RNA (or derivative thereof, e.g., inhibitory derivative) that are capable of inhibiting the different expressed genes can be placed into individual wells positioned on a microtiter plate as an ordered array, and intact cells/organisms in each well can be assayed for any changes or modifications in behavior or development due to inhibition of target gene activity. The amplified RNA can be fed directly to, injected into, the cell/organism containing the target gene. Alternatively, the IRE RNA (or derivative thereof, e.g., inhibitory derivative) can be produced from a vector, as described herein. Vectors can be injected into, the cell/organism containing the target gene. The function of the target gene can be assayed from the effects it has on the cell/organism when gene activity is inhibited. This screening could be amenable to small subjects that can be processed in large number, for example: arabidopsis, bacteria, drosophila, fungi, nematodes, viruses, zebrafish, and tissue culture cells derived from mammals. A nematode or other organism that produces a colorimetric, fluorogenic, or luminescent signal in response to a regulated promoter (e.g., transfected with a reporter gene construct) can be assayed in an HTS format.

D. Viral Delivery Vehicles

One challenge that must be met to realize therapeutic applications of RNAi technologies is the development of systems to deliver RNA agents efficiently into mammalian cells. One limitation of plasmid-based delivery systems is their dependence on cell transfection methods, which are often not efficient and are limited primarily to established cell lines. Viral based strategies would offer the significant advantage of allowing for efficient delivery to cell lines as well as primary cells. Recently, a retrovirus was designed to generate siRNAs driven from a pol-III dependent H1 promoter (Barton & Medzhitov (2002) PNAS 99:14943-45). Using this strategy, however, the integration of a high-copy number of the HI cassette into the host cell genome was required for efficient RNAi to be induced. A more efficient delivery system is clearly needed in the art.

Towards that end, cassettes or vectors can be designed for expressing RNAi agents. A preferred cassette or vector of the invention includes IRE sequences and/or sequences located adjacent to said IRE sequences that facilitate expression of said IRE RNA. In one embodiment, a preferred cassette or vector of the invention encodes a RNA derived from an IRE locus (e.g., SINE Alu element), wherein the RNA is initially processed by Drosha to a form accessible to other RNAi machinery, e.g., Dicer. In one embodiment, a preferred cassette or vector of the invention encodes a RNA derived from an IRE locus (e.g., SINE Alu element) and having a short hairpin or stem-loop structure that is processed by Dicer (or an orthologue or homologue thereof). The RNA derived from an IRE locus, e.g., short hairpin or stem-loop structures, are processed to generate siRNA- or mi-RNA-like molecules in cells or organisms and thereby induce gene silencing. In one embodiment, the sequences encoding the stem of the stem-loop structure are substituted with a designed sequence to produce a modified IRE RNA (e.g., modified to increase complementarity to a target RNA), which is then processed by cells (e.g., by Drosha and/or Dicer) to generate siRNA- or miRNA-like molecules which, in turn, induce gene silencing.

The siRNA- or miRNA-like molecules generated from IRE sequences of the invention may mediate posttranscriptional gene silencing, e.g., by inducing degradation of target RNA sequences or by inhibiting translation of target RNA sequences. The siRNA- or miRNA-like molecules generated from IRE sequences of the invention may also mediate transcriptional gene silencing, e.g., by inducing chromatin silencing at a target DNA sequence, wherein the target DNA sequence or sequences flanking the target DNA sequence encode a RNA to which the siRNA- or miRNA-like molecule is sufficiently complementary.

In one embodiment, expression of the RNA, e.g., short hairpin or stem-loop structure, is driven by a RNA polymerase III (pol III) promoters (T. R. Brummelkamp et al. Science (2002) 296:550-553; P. J. Paddison et al., Genes Dev. (2002) 16:948-958). Pol III promoters are advantageous because their transcripts are not necessarily post-transcriptionally modified, and because they are highly active when introduced in mammalian cells. Polymerase II (pol II) promoters may offer advantages to pol III promoters, including being more easily incorporated into viral expression vectors, such as retroviral and adeno-associated viral vectors, and the existence of inducible and tissue specific pol II dependent promoters.

In the instant invention, IRE loci are used to express miRNA- and siRNA-like molecules in cells and organisms. An IRE locus (e.g. Alu SINE locus) can be constructed to generate a short dsRNA sequence, e.g. ˜21-2 nt, having an intervening stem loop, that, when processed by Dicer, bears complementarity to a target RNA sequence. An IRE locus so constructed may produce a RNA that is initially processed by Drosha to a form accessible to Dicer, whereby subsequent processing by Dicer generates a short dsRNA sequence, e.g. ˜21-2 nt, that bears complementarity to a target RNA sequence. Vectors so modified could be highly efficient siRNA transduction systems. Also within the scope of the present invention are cassettes providing siRNA- or miRNA-like molecules similarly derived from IRE RNA or IRE RNA-like sequences/structures for the production of molecules with RNAi inducing activity, wherein the cassettes are present within other vectors or expression systems.

IREs (e.g., SINES, LINES, LTR-retrotransposons, and the like) are highly abundant in eukaryotic genomes, where, for example, the copy number of a single SINE element may exceed 10⁶. IREs are also predominantly located in untranslated regions of the genome. Accordingly, vectors and cassettes of the invention are particularly useful for achieving constitutive expression of miRNA- and siRNA precursors (e.g., short dsRNA sequence, e.g. ˜21-2 nt, having an intervening stem loop, that, when processed by Dicer, bears complementarity to a target RNA sequence) from IRE or IRE-like sequences in cells or organisms. More specifically, vectors and cassettes of the invention are useful for achieving genomic integration of IRE or IRE-like sequences (e.g., into mammalian cells and/or organisms) by targeting integration (e.g., via recombination) to homologous genomic IRE sequences. Such homologous genomic IRE sequences are preferably present in untranslated regions of the genome. Regulation of gene expression using vectors and cassettes as described herein offers significant advantages over current gene therapy methodologies. For example, the abundance of IRE loci in eukaryotic genomes provides significant opportunity for successful recombination and integration of IRE or IRE-like sequences into the genome. Moreover, targeting integration to untranslated regions of the genome is preferably to current gene therapy methodologies, wherein the integration of foreign DNA into coding regions of the genome of a subject can lead to undesirable effects.

V. Methods of Treatment

The present invention provides methods for identifying IRE RNAs and their targets (as well as modulators of said targets), which can further be used clinically (e.g., in certain prophylactic and/or therapeutic applications). For example, IRE RNAs can be used as prophylactic and/or therapeutic agents in the treatment of diseases or disorders associated with unwanted or aberrant expression of the corresponding target gene.

In one embodiment, the invention provides for prophylactic methods of treating a subject at risk of (or susceptible to) a disease or disorder for example, a disease or disorder associated with aberrant or unwanted target gene expression or activity. Subjects at risk for a disease which is caused or contributed to by aberrant or unwanted target gene expression or activity can be identified by, for example, any or a combination of diagnostic or prognostic assays as described herein. Administration of a prophylactic agent can occur prior to the manifestation of symptoms characteristic of the target gene aberrancy, such that a disease or disorder is prevented or, alternatively, delayed in its progression.

In another embodiment, the invention provides for therapeutic methods of treating a subject having a disease or disorder, for example, a disease or disorder associated with aberrant or unwanted target gene expression or activity. In an exemplary embodiment, the modulatory method of the invention involves contacting a cell capable of expressing target gene with a therapeutic agent that is specific for the target gene or protein (e.g., is specific for the mRNA encoded by said gene or specifying the amino acid sequence of said protein) such that expression or one or more of the activities of target protein is modulated. These modulatory methods can be performed in vitro (e.g., by culturing the cell with the agent) or, alternatively, in vivo (e.g., by administering the agent to a subject). As such, the present invention provides methods of treating an individual afflicted with a disease or disorder characterized by aberrant or unwanted expression or activity of a target gene polypeptide or nucleic acid molecule. Inhibition of target gene activity is desirable in situations in which target gene is abnormally unregulated and/or in which decreased target gene activity is likely to have a beneficial effect.

“Treatment”, or “treating” as used herein, is defined as the application or administration of a prophylactic or therapeutic agent to a patient, or application or administration of a prophylactic or therapeutic agent to an isolated tissue or cell line from a patient, who has a disease or disorder, a symptom of disease or disorder or a predisposition toward a disease or disorder, with the purpose to cure, heal, alleviate, relieve, alter, remedy, ameliorate, improve or affect the disease or disorder, the symptoms of the disease or disorder, or the predisposition toward disease.

In one embodiment, a disease or disorder is caused by or associated with the presence of (e.g., the insertion of, constitutive exonization of) an interspersed repetitive element (e.g., retrotransposable element) in a gene. For example, a disease or disorder may be caused by or associated with the constitutive exonization of an Alu intron. More than 5% of human alternatively spliced exons are Alu-derived, and most Alu-containing exons are alternatively spliced. While Alu-containing exons (being alternatively spliced) add a splice variant, there is always another messenger RNA without the Alu element in the coding region, thus maintaining the original protein intact. When the splicing of an Alu exon becomes constitutive, the transcript encoding the original protein is permanently disrupted, providing the basis for a genetic disorder. Mutations causing a constitutive splicing of intronic Alus are known to cause genetic diseases. For example, a point mutation in an Alu element residing in the third intron of the ornithine aminotransferase gene has been shown to activate a cryptic splice site, consequently leading to the introduction of a partial Alu element into an open reading frame; the in-frame stop codon carried by the Alu element results in a truncated protein and ornithine aminotransferase haplodeficiency (G. A. Mitchell et al., 1991 Proc. Natl. Acad. Sci. U.S.A. 88: 815). A mutation in the COL4A3 gene activates a constitutive exonization of a silent intronic Alu, resulting in Alport syndrome (B. Knebelmann et al., 1995 Hum. Mol. Genet. 4: 675). Recent studies revealed that alternative splicing of Alu exons can be regulated by a single point mutation (G. Lev-Maor et al. 2003 Science 300: 1288) and suggest that many silent intronic Alu elements are susceptible to exonization, providing a molecular basis for predisposition to so-far uncharacterized genetic diseases. Therapeutic methods of the invention are particularly useful for a disease or disorder in which the constitutive splicing of an Alu alternatively spliced exon results in a gain-of-function mutation.

In one embodiment, a target gene of the invention is an antiviral target. In another embodiment, a target gene of the invention is a gene involved in maintaining cellular homeostasis. Examples of genes involved in maintenance of homeostasis include, for example, genes associated with regulation of cell growth, including growth factors or receptors for growth factors, transcription factors, apoptotic or anti-apoptotic factors, and tumor suppressor genes. In another embodiment, a target gene of the invention is a gene involved in maintenance of differentiation or regulation of glucose metabolism. Modulation of such genes is particularly useful, for example, to treat any of a number of disorders (including cancer, inflammation, neuronal disorders, etc.). In another embodiment, a target gene of the invention is a gene comprising an IRE (e.g., Alu element), or portion thereof. Examples of genes comprising an IRE (e.g., Alu element) or portion thereof are genes having, e.g., an Alu intron, an alternatively spliced Alu exon, or a constitutively spliced Alu exon.

Further, since miRNAs are believed to be involved in translational control, knowledge of miRNA-like molecules and their targets would allow specific modulation of a variety of systems controlled at the translational level. Manipulating translation of genes (e.g., the genes described above) is a novel, powerful, and specific method for treating these disorders.

The present invention further contemplates the use of IRE RNAs (and derivatives thereof) as well as modulators, for example, of IRE RNA targets, in various agricultural treatments. In one embodiment, a compound or agent of the invention is used to modulate RNAi in an insect. In another embodiment, a compound or agent of the invention is used to modulate RNAi in a bacteria. In another embodiment, a compound or agent is used to modulate RNAi in a parasite. In certain embodiments, a compound or agent is administered to the organism (e.g., fed to the organism). In certain embodiments, the organism ingests the compound or agent. An exemplary compound or agent makes the organism sterile upon ingestion. In another embodiment, a compound or agent of the invention is used to modulate RNAi in a plant.

VI. Pharmacogenomics and Pharmaceutical Compositions

With regards to both prophylactic and therapeutic methods of treatment, such treatments may be specifically tailored or modified, based on knowledge obtained from the field of pharmacogenomics. “Pharmacogenomics”, as used herein, refers to the application of genomics technologies such as gene sequencing, statistical genetics, and gene expression analysis to drugs in clinical development and on the market. More specifically, the term refers the study of how a patient's genes determine his or her response to a drug (e.g., a patient's “drug response phenotype”, or “drug response genotype”). Thus, another aspect of the invention provides methods for tailoring an individual's prophylactic or therapeutic treatment with either the target gene molecules of the present invention or target gene modulators according to that individual's drug response genotype. Pharmacogenomics allows a clinician or physician to target prophylactic or therapeutic treatments to patients who will most benefit from the treatment and to avoid treatment of patients who will experience toxic drug-related side effects.

With regards to the above-described agents for prophylactic and/or therapeutic treatments (e.g., IRE RNAs or derivatives thereof), the agents are routinely incorporated into pharmaceutical compositions suitable for administration. Such compositions typically comprise the nucleic acid molecule, protein, antibody, or modulatory compound and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, intraperitoneal, intramuscular, oral (e.g., inhalation), transdermal (topical), and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated: each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit large therapeutic indices are preferred. Although compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the EC50 (i.e., the concentration of the test compound which achieves a half-maximal response) as determined in cell culture. Such information can be used to more accurately determine useful doses in humans. Levels in plasma may be measured, for example, by high performance liquid chromatography.

When administering IRE RNAs (or derivatives thereof), it may be advantageous to chemically modify the RNA in order to increase in vivo stability. Preferred modifications stabilize the RNA against degradation by cellular nucleases.

The pharmaceutical compositions can be included in a container, pack, or dispenser together with instructions for administration.

This invention is further illustrated by the following examples which should not be construed as limiting. The contents of all references, patents and published patent applications cited throughout this application are incorporated herein by reference.

### EXAMPLES

### Example I

### Northern Analysis of Alu RNA Cleavage Products in Heat Shocked or Adenovirus Infected Cells

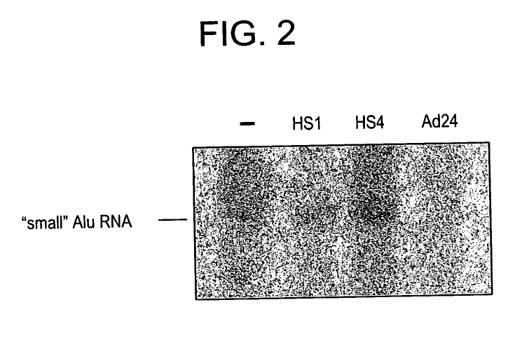

To test whether Alu RNAs are in fact processed into small RNAs under normal or stressed conditions, HeLa cells were subjected to heat shock or adenovirus infection and the presence of Alu RNA cleavage products was examined by Northern analysis. Conditions for heat shock and adenovirus infection were essentially as described (Li and Schmid, 2001 Gene 276:135-141). Briefly, to induce heat shock stress, HeLa cells were incubated at 45° C. for 30 minutes and then returned to 37° C. Cells were harvested for RNA at 1 hr (HS1) and 4 hrs (HS4) post heat shock. Alternately, cells were infected with adenovirus (MOI=5) and RNA isolated at 24 hrs post infection (Ad24). RNA was extracted using Trizol reagent (Invitrogen) according to the manufacturer's protocol. 25 μg of each sample was electrophoresed on a 15% PAGE gel under denaturing conditions, and the gel was transferred to a nylon membrane via semi-dry electroblotting at 400 mA for one hour. RNA was crosslinked to the nylon membrane by UV crosslinking (Stratagene, Stratalinker). The membrane was pre-hybridized for 1 hr at 37° C. in a Church's buffer and then hybridized overnight with a combination of three non-overlapping, radiolabeled probes (25 pmols each), which are complementary to the ascending stem of the first Alu stem-loop, the loop of the second stem-loop, and the descending strand of the second stem-loop of Alu. Results of the experiment are presented in FIG. 2. The region of the Northern image where RNAs in the range of 15-25 nt migrated is shown. (−) denotes untreated HeLa cells. The results demonstrate that Alu RNA is processed into one or more small RNAs and that the levels of these small RNAs increase at 4 hrs after heat shock induction. The results also suggest that adenovirus infection may inhibit this processing.

### Example II

### Analysis of IRE RNA Cleavage in Drosophila Embryo Extract and by Recombinant Dicer and/or Drosha

Drosophila embryo extracts competent for Dicer cleavage are incubated for various times with ³²P-labeled IRE RNA, e.g., Alu RNA, or pre-Let-7 precursor substrates to test potential cleavage of IRE RNA by Dicer. Pre-Let-7 is known to be processed to ˜22nt product in this reaction, and thus serves as a positive control. Reactions can be performed essentially as described (see Tuschl et al, Genes Dev (1999), 13:3191-97) and under conditions favorable for cleavage of IRE RNA. Reaction products are then deproteinated and analyzed on a PAGE gel (Tuschl et al, 1999). Cleavage products of similar size to those generated by cleavage of the pre-Let-7 substrate are evidence that an activity in the Drosophila embryo extract is able to recognize and cleave the IRE RNA in a manner similar to the processing of the known miRNA precursor, pre-Let-7.

Using the same templates as set forth above, reactions are also carried out with recombinant Dicer enzyme (Gene Therapy Systems) to analyze potential recognition and cleavage of IRE RNAs, e.g., Alu RNA, by the purified enzyme. Reactions are performed essentially as described by the manufacturer. Reaction products are then deproteinated and analyzed on a PAGE gel. A negative control reaction is one in which template RNA is not subjected to the Dicer reaction. The accumulation of products of similar size to those generated in the Drosophila lysate (e.g., ˜21nt IRE RNA cleavage products) indicate that the activity in the lysate observed to cleave IRE RNA is likely that of Dicer. Time courses of IRE RNA cleavage using recombinant Dicer enzyme can also be carried out by scaling up the reactions and removing aliquots over time. Reaction products are analyzed as described above.

Using the same templates as set forth above, reactions are also carried out with Drosha enzyme (either highly purified from cell extracts or in recombinant form) to analyze potential recognition and cleavage of IRE RNAs, e.g., Alu RNA, by the enzyme. Reactions are performed under conditions favorable for Drosha activity and analyzed as described above.

### Example III

### Northern Analysis of IRE RNA Cleavage Products in Cells

Northern blot analyses are performed to detect 21-25 nt cleavage products derived from other IRE RNAs in addition to the Alu RNAs examined in Example I above, e.g., LINES or other SINES. Experiments are performed similarly as in Example I. Briefly, cells expressing IRE RNA are lysed in Trizol reagent (Invitrogen) according to the manufacturer's protocol. RNA from these cells is electrophoresed through a 15% PAGE gel under denaturing conditions, and the resolved nucleic acids transferred to a nylon membrane via semi-dry electroblotting. Included in this gel are Dicer-cleaved (and/or a combination of Drosha- and Dicer-cleaved) IRE RNA reactions which serve as positive controls for hybridization with probe. Electroblotted RNA is then crosslinked to the nylon membrane by UV crosslinking (Stratagene, Stratalinker). The membrane is pre-hybridized for 1 hr at 37° C. in a formamide hybridization buffer and then hybridized overnight with full length probe for said IRE RNA (³²P-labeled reverse complement transcript of IRE RNA). Alternatively, ³²P-labeled oligonucleotides complementary to IRE RNA sequences can be used as probes for IRE RNA. The following day, the membrane is washed and bands are detected using a Phosphorimager. Detection of 21-25 nt fragments of IRE RNA is indicative of processing of IRE RNA into miRNA-like moieties in vivo.

### REFERENCES

1. B. N. Fields, D. M. Knipe, P. M. Howley, Fundamental Virology (Lippincott Williams & Wilkins, Philadelphia, Pa., ed. Third, 1996).
2. N. C. Lau, L. P. Lim, E. G. Weinstein, D. P. Bartel, Science 294, 858-62. (2001).
3. G. J. Hannon, Nature 418, 244-51. (2002).
4. G. Hutvagner, P. D. Zamore, Curr Opin Genet Dev 12, 225-32. (2002).
5. P. M. Waterhouse, M. B. Wang, T. Lough, Nature 411, 834-42. (2001).
6. O. Voinnet, Trends Genet 17,449-59. (2001).
7. H. Li, W. X. Li, S. W. Ding, Science 296, 1319-21. (2002).
8. C. E. Samuel, Clin Microbiol Rev 14, 778-809, table of contents (October, 2001).
9. S. M. Elbashir et al, Nature 411, 494-8. (2001).
10. M. T. McManus, P. A. Sharp, Nat Rev Genet 3, 737-47. (2002).
11. A. Grishok et al., Cell 106, 23-34. (2001).
12. M. Lagos-Quintana, R. Rauhut, W. Lendeckel, T. Tuschl, Science 294, 853-8. (2001).
13. M. W. Rhoades etal, Cell 110, 513-20. (2002).
14. G. Hutvagner, P. D. Zamore, Science 297, 2056-60. (2002).
15. C. Llave, Z. Xie, K. D. Kasschau, J. C. Carrington, Science 297, 2053-6. (2002).
16. J. M. Jacque, K. Triques, M. Stevenson, Nature 418, 435-8. (2002)
17. A. P. McCaffrey et al., Nature 418, 38-39. (2002).
18. T. Tuschl, Nat Biotechnol 20, 446-8. (2002)
19. G. Sui et al., Proc Natl Acad Sci USA 99, 5515-20 (2002)
20. G. M. Barton, R. Medzhitov, Proc Natl Acad Sci USA 99, 14943-5. (2002)
21. M. B. Mathews, T. Shenk, J. Virol 65, 5657-62. (1991).
22. Y. Ma, M. B. Mathews, J Virol 70, 5083-99. (1996)
23. J. G. Howe, M. D. Shu, J Virol 62, 2790-8 (1988).
24. R. J. Bowden, J. P. Simas, A. J. Davis, S. Efstathiou, J Gen Virol 78 (Pt7), 1675-87 (1997).
25. R. Marschalek et al., Nucleic Acids Res 17, 631-43 (1989).
26. J. H. chen, S. Y. Le, J. V. Maizel, Nucleic Acids Res 28, 991-9 (2000).
27. L. Bieleski, S. J. Talbot, J Virol 75, 1864-9 (2001)
28. K. Ochs et al., J Virol 76, 2113-22 (2002).
29. C. M. Spahn et al., Science 291, 1959-62 (2001).
30. Y. Lee et al., Nature 425, 415-419 (2003).
31. M. Matzke and A. J. M. Matzke, Science 301, 1060-1061 (2003).
32. M. A. Matzke et al., Curr. Opin. Gen. Dev.11, 221-227 (2003).

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for identifying a druggable target, comprising:
(a) obtaining an assay composition comprising an RNAi pathway molecule and an interspersed repetitive element (IRE) RNA;
(b) assaying for expression of a candidate RNA;
wherein a change in expression of the candidate RNA indicates that a gene or protein corresponding to the RNA is a druggable target.

2. The method of claim 1, wherein the assay composition is a cell extract.

3. The method of claim 1, wherein the assay composition is a mammalian cell extract.

4. A method for identifying a druggable target, comprising:
(a) obtaining a cell or organism comprising an RNAi pathway and an interspersed repetitive element (IRE) RNA;
(b) assaying for expression of a candidate RNA;
wherein a change in expression of the candidate RNA indicates that a gene or protein corresponding to the RNA is a druggable target.

5. The method of claim 1 or 4, wherein the druggable target in an antiviral drug target.

6. The method of claim 4, wherein the cell is a eukaryotic cell.

7. The method of claim 4, wherein the cell is a plant cell.

8. The method of claim 4, wherein the cell is an insect cell.

9. The method of claim 4, wherein the cell is a mammalian cell.

10. The method of claim 4, wherein the cell is a murine cell.

11. The method of claim 4, wherein the cell is an avian cell.

12. The method of claim 4, wherein the cell is a human cell.

13. The method of any one of the preceding claims, wherein the change in expression of the candidate RNA is a decrease in the expression of the candidate RNA

14. The method of any one of the preceding claims, further comprising the step of preselecting the candidate RNA.

15. The method of claim 14, wherein the preselection step comprises determining a sufficient degree of sequence identity between the interspersed repetitive element (IRE) RNA and the candidate RNA.

16. The method of claim 15, wherein the IRE RNA and the candidate RNA share at least 60% sequence identity

17. The method of claim 15, wherein the IRE RNA and the candidate RNA share at least 70% sequence identity

18. The method of claim 15, wherein the IRE RNA and the candidate RNA share at least 80% sequence identity

19. The method of claim 15, wherein the IRE RNA and the candidate RNA share at least 90% sequence identity.

20. The method of claim 14, wherein the preselection step comprises selecting the candidate RNA based on its encoding a gene or protein having a desired cellular function.

21. The method of claim 20, wherein the desired cellular function is selected from the group consisting of maintenance of cellular homeostasis, maintenance of differentiation, regulation of cell cycle, regulation of glucose metabolism, promotion of apoptosis and inhibition of apoptosis.

22. The method of claim 14, wherein the preselection step comprises selecting the candidate RNA based on its comprising an interspersed repetitive element (IRE) sequence or portion thereof.

23. The method of any one of claims 1-22, wherein the candidate RNA is a mRNA.

24. The method of any one of claims 1-22, wherein the candidate RNA encodes a cellular protein.

25. The method of any one of claims 1-22, wherein the candidate RNA encodes a viral protein.

26. The method of any one of claims 1-22, wherein the candidate RNA is a ncRNA regulating gene expression.

27. The method of any one of claims 1-22, wherein the candidate RNA is transcribed from a gene comprising an interspersed repetitive element (IRE) or portion thereof.

28. The method of any one of the preceding claims, wherein the interspersed repetitive element is selected from the group consisting of a short interspersed element (SINE), a long interspersed element (LINE), and a long terminal repeat (LTR)-retrotransposon.

29. The method of any one of claims 1-28, wherein the interspersed repetitive element is a LTR-retrotransposon.

30. The method of any one of claims 1-28, wherein the interspersed repetitive element is a long interspersed element (LINE).

31. The method of any one of claims 1-28, wherein the interspersed repetitive element is a short interspersed element (SINE).

32. The method of claim 31, wherein the short interspersed element is an Alu element.

33. The method of any one of claims 1 to 32, wherein the interspersed repetitive element RNA is expressed from a virus.

34. The method of any one of claims 1 to 32, wherein the interspersed repetitive element RNA is expressed from a vector.

35. The method of any one of claims 1-32, wherein the interspersed repetitive element RNA is expressed from a cassette.

36. A druggable target identified according to any one of claims 1-35.

37. A method for identifying a therapeutic agent, comprising assaying a test agent for activity against the druggable target of claim 36.

38. A method for identifying a therapeutic agent, comprising assaying a test agent for the ability to stimulate expression or activity of the druggable target of claim 36.

39. A method for identifying a therapeutic agent, comprising assaying a test agent for the ability to inhibit an interaction between the druggable target of claim 36 and a corresponding interspersed repetitive element RNA.

40. A method for identifying a therapeutic agent, comprising:
(a) contacting a cell with a test agent, said cell comprising an RNAi pathway and an interspersed repetitive element RNA, wherein said RNAi pathway generates a siRNA or miRNA from said interspersed repetitive element RNA;
(b) detecting an indicator of said siRNA or miRNA;
wherein an agent is identified based on its ability to inhibit the generation of said siRNA or miRNA.

41. A method for identifying a therapeutic agent, comprising:
(a) contacting an assay composition with a test agent, wherein said assay composition comprises an RNAi pathway molecule and an IRE RNA, wherein said RNAi pathway molecule generates a siRNA or miRNA from said IRE RNA;
(b) detecting an indicator of said siRNA or miRNA;
wherein an agent is identified based on its ability to inhibit the generation of said siRNA or miRNA.

42. An agent identified by the method of any one of claims 37-41.

43. A composition comprising the agent of claim 42 and a pharmaceutically acceptable carrier.

44. A method of treating a disease or disorder in a subject, comprising administering to the subject a therapeutically effective dose of the agent of claim 42 or the composition of claim 43, such that the disease or disorder is treated.

45. The method of claim 44, wherein the organism or subject is a eukaryotic organism.

46. The method of claim 44, wherein the organism or subject is a mammal.

47. The method of claim 44, wherein the organism or subject is a human.

48. A method of inhibiting RNAi in a cell, comprising introducing into the cell an interspersed repetitive element (IRE) RNA or inhibitory derivative thereof, such that RNAi in the cell is inhibited.

49. A method of inhibiting the incorporation of a siRNA or miRNA into a cellular Dicer or RISC complex, comprising introducing into the cell an isolated interspersed repetitive element (IRE) RNA or inhibitory derivative thereof, such that incorporation of the siRNA or miRNA into the complex is inhibited.

50. The method of claim 48 or 49, wherein the cell is a eukaryotic cell.

51. The method of claim 48 or 49, wherein the cell is a mammalian cell.

52. The method of claim 48 or 49, wherein the cell is a human cell.

53. The method of any one of claims 48-52, wherein the cell is present in an organism.

54. The method of claim 53, wherein the cell is present in a human subject.

55. The method of any one of claims 48-54, wherein the IRE is a long terminal repeat (LTR)-retrotransposon.

56. The method of any one of claims 48-54, wherein the IRE is a long interspersed element (LINE).

57. The method of any one of claims 48-54, wherein the IRE is a short interspersed element (SINE).

58. The method of claim 57, wherein the SINE is an Alu element.

59. The method of any one of claims 48-54, wherein the IRE RNA is expressed from a virus.

60. The method of any one of claims 48-54, wherein the IRE RNA is expressed from a vector.

61. The method of any one of claims 48-54, wherein the IRE RNA is expressed from a cassette.

62. A method for identifying a therapeutic agent, comprising:
(a) contacting a cell with a test agent, said cell comprising an RNAi pathway and an interspersed repetitive element (IRE) RNA, wherein the ribonucleotide inhibits the RNAi pathway;
(b) detecting an indicator of the RNAi pathway;
wherein an agent is identified based on its ability to promote inhibition of the RNAi pathway.

63. A method for identifying a therapeutic agent, comprising:
(a) contacting an assay composition with a test agent, wherein said assay composition comprises a RNAi pathway molecule and an interspersed repetitive element (IRE) RNA which inhibits the activity of said RNAi pathway molecule;
(b) detecting activity of said RNAi pathway molecule;
wherein said agent is identified based on its ability to further inhibit activity of said RNAi pathway molecule.

64. A method for identifying a therapeutic agent, comprising:
(a) contacting an assay composition with a test agent, wherein said assay composition comprises an interspersed repetitive element (IRE) RNA and a RNAi pathway molecule capable of interacting with or altering the IRE RNA;
(b) detecting the ability of the RNAi pathway molecule to interact with or alter the IRE RNA;
wherein said agent is identified based on its ability to modulate the interaction of the IRE RNA with RNAi pathway molecule or alteration of the IRE RNA by the RNAi pathway molecule.

65. The method of claim 63 or claim 64, wherein the RNAi pathway molecule is a RISC component.

66. The method of claim 63 or claim 64, wherein the RNAi pathway molecule is Dicer, or a homologue thereof.

67. An agent identified according to the method of any one of claims 62-66.

68. A composition comprising the agent of claim 67 and a pharmaceutically acceptable carrier.

69. A vector for delivering a siRNA or miRNA, comprising an interspersed repetitive element (IRE) locus that has been modified to comprise a nucleotide sequence that encodes a siRNA or miRNA precursor.

70. A cassette for expressing a siRNA or miRNA, comprising an interspersed repetitive element (IRE) locus that has been modified to comprise a nucleotide sequence that encodes a siRNA or miRNA precursor.

71. The vector of claim 69 or cassette of claim 70, further comprising a polymerase III promoter operably linked to the nucleotide sequence.

72. The vector of claim 69 or cassette of claim 70, further comprising a promoter endogenous to the IRE locus operably linked to the nucleotide sequence.

73. The vector or cassette of any one of claims 69-72, wherein the sequence of the miRNA or siRNA molecule is sufficiently complementary to a RNA sequence to mediate degradation of said RNA sequence.

74. The vector or cassette of any one of claims 69-72, wherein the sequence of the miRNA molecule is sufficiently complementary to a RNA sequence to inhibit translation of said RNA sequence.

75. The vector or cassette of any one of claims 69-72, wherein the sequence of the miRNA molecule is sufficiently complementary to a RNA sequence to induce chromatin silencing of a DNA sequence encoding the RNA sequence.

76. The vector or cassette of any one of claims 69-75, wherein the IRE is a long terminal repeat (LTR)-retrotransposon.

77. The vector or cassette of any one of claims 69-75, wherein the IRE is a long interspersed element (LINE).

78. The vector or cassette of any one of claims 69-75, wherein the IRE is a short interspersed element (SINE).

79. The vector or cassette of claim 78, wherein the SINE is an Alu element.

80. The vector of claim 69, wherein the vector is a plasmid.

81. The vector of claim 69, wherein the vector is derived from a virus.

82. A vector that expresses a siRNA or miRNA from an interspersed repetitive element (IRE) locus.

83. The vector of claim 82, wherein the siRNA or miRNA is exogenous.

84. A composition comprising the vector of claim 82 or 83 and a pharmaceutically acceptable carrier.

85. A method for targeting degradation of a RNA in a subject, comprising administering to the subject the composition of claim 84, wherein the siRNA or miRNA has a ribonucleotide sequence having sufficient complementarity to the target RNA, such that the targets are degraded.

86. The method of claim 85, wherein the siRNA or miRNA has a ribonucleotide sequence sufficiently complementary to a mutant allelic target RNA, such that the mutant allelic target is degraded.

87. A method for targeting a RNA for translational inhibition in a subject, comprising administering to the subject the composition of claim 84, wherein the siRNA or miRNA has a ribonucleotide sequence having sufficient complementarity to the target RNA, such that the targets are translationally inhibited.

88. The method of claim 87, wherein at least one siRNA or miRNA has a ribonucleotide sequence sufficiently complementary to a mutant allelic target RNA, such that the mutant allelic target is translationally inhibited.

89. A method for targeting a DNA sequence for chromatin silencing in a subject, comprising administering to the subject the composition of claim 84, wherein the siRNA or miRNA has a ribonucleotide sequence having sufficient complementarity to a RNA encoded by the target DNA sequence such that the target DNA sequence is chromatically silenced.

90. The method of claim 89, wherein at least one siRNA or miRNA has a ribonucleotide sequence sufficiently complementary to a RNA encoded by a mutant allelic target DNA sequence, such that the mutant allelic target DNA sequence is chromatically silenced.

91. The method of any one of claims 85-90, wherein the interspersed repetitive element (IRE) RNA locus becomes integrated in the genome of the subject.

92. The method of claim 91, wherein integration is at a genomic IRE locus.

93. The method of claim 92, wherein the genomic IRE locus is present in an untranslated region of the genome.

94. A vaccine comprising the vector of claim 82 or 83, wherein at least one siRNA or miRNA targets a viral gene product.

95. A vaccine comprising the vector of claim 82 or 83, wherein at least one siRNA or miRNA targets a cellular gene.

96. A method for upregulating exogenous gene expression in a cell, comprising introducing into a cell having an RNAi pathway an interspersed repetitive element (IRE) RNA, wherein the IRE RNA is a substrate or inhibitor of the RNAi pathway, such that exogenous gene expression is upregulated.

97. A method for efficiently introducing an exogenous gene into a cell, comprising introducing into a cell having an RNAi pathway the exogenous gene and an interspersed repetitive element (IRE) RNA, wherein the IRE RNA is a substrate or inhibitor of the RNAi pathway, such that the exogenous gene is efficiently introduced.

98. The method of claim 96 or 97, wherein the cell is a eukaryotic cell.

99. The method of claim 96 or 97, wherein the cell is a mammalian cell.

100. The method of claim 96 or 97, wherein the cell is a human cell.

101. The method of any one of claims 96-100, wherein the cell is present in an organism.

102. The method of claim 101, wherein the cell is present in a human subject.

103. The method of claims 96-102, wherein the interspersed repetitive element is selected from the group consisting of a short interspersed element (SINE), a long interspersed element (LINE), and a long terminal repeat (LTR)-retrotransposon.

104. The method of any one of claims 96-103, wherein the interspersed repetitive element RNA is expressed from a virus.

105. The method of any one of claims 96-103, wherein the interspersed repetitive element RNA is expressed from a vector.

106. The method of any one of claims 96-103, wherein the interspersed repetitive element RNA is expressed from a cassette.
